(19) United States
(12) Patent Application Publication
(10) Pub. No.: US 2004/0209776 A1
(43) Pub. Date: Oct. 21, 2004

(54) Azole derivatives useful as insecticide

(76) Inventors: Saleem Farooq, Basel (CH); Roger Graham Hall, Basel (CH); Christopher John Mathews, Bracknell (GB)

Correspondence Address: SYNGENTA CROP PROTECTION , INC., PATENT AND TRADEMARK DEPARTMENT, 410 SWING ROAD, GREENSBORO, NC, 27409, US

(21) Appl. No.: 10/484,988

(22) PCT Filed: Jul. 26, 2002
(86) PCT No.: PCT/GB02/03442

(30) Foreign Application Priority Data
Jul. 27, 2001 (GB) 0118357.3

(51) Int. Cl. A01N 43/40; A01N 43/42; A01N 43/78; C07D 417/02; A01N 43/80

(52) U.S. Cl. 504/244; 504/247; 504/267; 546/159; 546/270.1; 548/152; 548/206; 504/269

(57) ABSTRACT

This invention relates to improved azole derivatives of formula (I): where B is O or S; Het is a heterocycle selected from a number of specified heterocycles and R¹, R², R³, R⁴, R⁵, R⁶, R⁷, R⁸, R⁹ and R¹⁰ are each selected from specified groups; provided that at least one of R⁶ and R¹⁰ is not hydrogen; and to insecticidal, acaricidal, molluscicidal and nematicidal compositions comprising them and to methods of using them to combat and control insect, acarine, mollusc and nematode pests.

The present invention relates to improved azole derivatives, to insecticidal, acaricidal, molluscicidal and nematicidal compositions comprising them and to methods of using them to combat and control insect, acarine, mollusc and nematode pests.

Azole and azine derivatives are disclosed in WO95/31448, WO97/18198, WO98/02424, WO98/05670 and WO98/17630. In WO00/06566 there are disclosed insecticidal azole derivatives.

The applicants have found a group of compounds showing advantages over the compounds disclosed in WO00/06566. The present invention therefore provides a compound of formula (I):

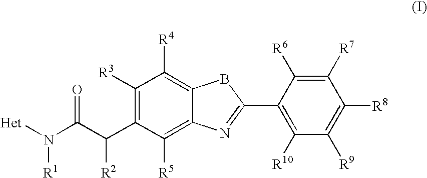

where B is O or S; Het is a heterocycle selected from heterocycles (a), (b), (c), (d), (e), (f), (g) and (h) in each of which the arrow shows the point of attachment to N of formula (I);

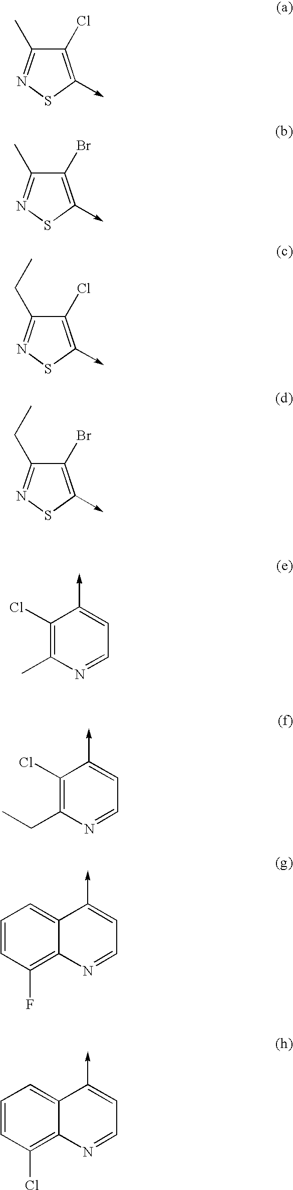

R¹ is hydrogen, C₁₋₂ alkyl, (C₁₋₆)alkoxymethyl or propargyl; R² is hydrogen, methyl or fluoro; R³, R⁴ and R⁵ are, independently, hydrogen, halogen, C₁₋₂ alkyl, C₁₋₂ alkoxy or C₁₋₂ haloalkyl; R⁶ and R¹⁰ are, independently, hydrogen, halogen, C₁₋₃ alkyl, C₁₋₂ haloalkyl, C₁₋₂ alkoxy, nitro, cyano, C₁₋₂ haloalkoxy, C₁₋₈ alkylthio, C₁₋₆ alkylsulfinyl, C₁₋₆ alkylsulfonyl, amino, C₁₋₃ alkylamino or di(C₁₋₃)alkylamino, provided that at least one of R⁶ and R¹⁰ is not hydrogen; R⁷, R⁸ and R⁹ are, independently, hydrogen, halogen, C₁₋₆ alkyl, C₂₋₆ alkenyl, C₂₋₆ alkynyl, C₁₋₆ haloalkyl, C₁₋₆ alkoxy(C₁₋₆)alkyl, C₁₋₆ alkoxy, C₁₋₆ alkoxy(C₁₋₆)alkoxy, C₂₋₆ alkynyloxy, C₃₋₆ cycloalkyl, nitro, cyano, C₁₋₆ haloalkoxy, C₂₋₆ haloalkenyloxy, S(O)ₚR¹¹, OSO₂R¹², NR¹³SO₂R¹⁴, NR¹⁵R¹⁶, NR¹⁷COR¹⁸, COR¹⁹, SiR²⁰R²¹R²², SCN, optionally substituted aryl or optionally substituted heteroaryl; R¹¹, R¹² and R¹⁴ are, independently, C₁₋₆ alkyl, C₁₋₆ haloalkyl or optionally substitituted aryl; R¹³ and R¹⁷ are, independently, hydrogen or C₁₋₂ alkyl; R¹⁵ and R¹⁶ are, independently, hydrogen or C₁₋₃ alkyl; or R¹⁵ and R¹⁶ together with the N atom to which they are attached form a five or six-membered optionally substituted heterocyclic ring which may contain a further heteroatom selected from O and S; R¹⁸ and R¹⁹ are, independently, hydrogen, C₁₋₆ alkyl, C₁₋₆ alkoxy, optionally substituted aryl, optionally substituted heteroaryl or NR²³R²⁴; R²⁰, R²¹ and R²² are, independently, C₁₋₄ alkyl or aryl; R²³ and R²⁴ are, independently, hydrogen or C₁₋₃ alkyl; or R²³ and R²⁴ together with the N atom to which they are attached form a five or six-membered optionally substituted heterocyclic ring which may contain a further heteroatom selected from O and S; and p is 0, 1 or 2; provided that when Het is a heterocycle selected from heterocycles (a), (b), (c) and (d); and R¹ is hydrogen, C₁₋₂ alkyl, (C₁₋₂)alkoxymethyl or propargyl; and R² is hydrogen; and R³, R⁴ and R⁵ are each hydrogen; then the moiety (M) where the arrow shows the point of attachment to the benzo-fused ring system of formula (I)

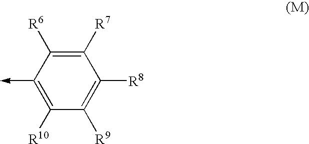

is not 2-Br—C₆H₄, 2-Cl—C₆H₄, 2,3-diCl—C₆H₃, 2,4-diCl—CH₆H₃, 2,5-diCl—C₆H₃, 2,6-diCl—C₆H₃, 2,4,6-triCl—C₆H₂, C₆Cl₅, 2-Cl-4-F—C₆H₃, 2-Cl-6-F—C₆H₃, 4-Cl-2,5-diF—C₆H₂, 2-Cl-4-NO₂—C₆H₃, 2-Cl-4-CF₃—C₆H₃, 2-Cl-6-CF₃—C₆H₃, 2-Cl-4-methanesulfonyl-C₆H₃, 2,4-diCl-5-F—C₆H₂, 2-F—C₆H₄, 2,3-diF—C₆H₃, 2,4-diF—C₆H₃, 2,5-diF—C₆H₃, 2,6-diF—C₆H₃, 2,3,4-triF—C₆H₂, 2,3,5-triF—C₆H₂, 2,3,6-triF—C₆H₂, 2,4,6-triF—C₆H₂, 2,3,4,5-tetraF—C₆H, C₆F₅, 2-F-3-CF₃—C₆H₃, 2-F-4-CF₃—C₆H₃, 2-F5-CF₃—C₆H₃,2-F-6-CF₃—C₆H₃, 4-F-2-CF₃—C₆H₃, 5-F-2-CF₃—C₆H₃, 2-CN—C₆H₄, 2-C₂H₅O—C₆H₄, 2-C₂H₅—C₆H₄, 2-CH₃O—C₆H₄, 2,6-diCH₃O—C₆H₃, 2—CH₃—C₆H₄, 2,3-diCH₃—C₆H₃, 2,4-diCH₃—C₆H₃, 2,5-diCH₃—C₆H₃, 2,6-diCH₃—C₆H₃, 2,4,6-triCH₃—C₆H₂, 2-NO₂—C₆H₄, 4-methanesulfonyl-2-nitrophenyl or 2-trifluoromethylphenyl.

The compounds of formula (I) may exist in different geometric or optical isomers or tautomeric forms. This invention covers all such isomers and tautomers and mixtures thereof in all proportions as well as isotopic forms such as deuterated compounds.

Each alkyl moiety is a straight or branched chain and is, for example, methyl, ethyl, n-propyl, n-butyl, n-pentyl, n-hexyl, isopropyl, n-butyl, sec-butyl, iso-butyl, tert-butyl or neo-pentyl.

Halogen is fluorine, chlorine, bromine or iodine.

Haloalkyl groups are alkyl groups which are substituted with one or more of the same or different halogen atoms and are, for example, CF₃, CF₂Cl, CF₃CH₂ or CHF₂CH₂.

Alkenyl and alkynyl moieties can be in the form of straight or branched chains. The alkenyl moieties, where appropriate, can be of either the (E)- or (Z)-configuration. Examples are vinyl, allyl, ethynyl and propargyl.

Haloalkenyl moieties are alkyl moieties which are substituted with one or more of the same or different halogen atoms, an example being CH₂CH═CCl₂. Aryl includes naphthyl, anthracyl, fluorenyl and indenyl but is preferably phenyl.

The term heteroaryl refers to an aromatic ring containing up to 10 atoms including one or more heteroatoms (preferably one or two heteroatoms) selected from O, S and N. Examples of such rings include pyridine, pyrimidine, furan, quinoline, quinazoline, pyrazole, thiophene, thiazole, oxazole and isoxazole.

Cycloalkyl includes cyclopropyl, cyclopentyl and cyclohexyl.

When present, the optional substituents on aryl or heteroaryl are selected, independently, from hydrogen, halogen, C₁₋₆ alkyl, C₂₋₆ alkenyl, C₂₋₆ alkynyl, C₁₋₆ haloalkyl, C₁₋₆ alkoxy(C₁₋₆)alkyl, C₁₋₆ alkoxy, C₃₋₆ cycloalkyl, nitro, cyano, C₁₋₆ haloalkoxy, C₁₋₂ alkylthio, SO₂CH₃, SO₂CH₂CH₃, OSO₂CH₃ and SCN.

It is to be understood that dialkylamino substituents include those where the dialkyl groups together with the N atom to which they are attached form a five, six or seven-membered heterocyclic ring which may contain one or two further heteroatoms selected from O, N or S and which is optionally substituted by one or two independently selected (C₁₋₆)alkyl groups. When heterocyclic rings are formed by joining two groups on an N atom, the resulting rings are suitably pyrrolidine, piperidine, thiomorpholine and morpholine each of which may be substituted by one or two independently selected (C₁₋₆) alkyl groups.

Preferably the optional substituents for cycloallyl include halogen, cyano and C₁₋₃ alkyl.

In one embodiment of the present invention, there is provided a compound of formula (I) as defined above where R⁶ and R¹⁰ are, independently, hydrogen, halogen, C₁₋₃ alkyl, C₁₋₂ haloalkyl, C₁₋₂ alkoxy, nitro, cyano, C₁₋₂ haloalkoxy, C₁₋₂alkylthio, amino, C₁₋₃ alkylamino or di(C₁₋₃)alkylamino, provided that at least one of R⁶ and R¹⁰ is not hydrogen; and R⁷, R⁹ and R⁹ are, independently, hydrogen, halogen, C₁₋₆ alkyl, C₂₋₆ alkenyl, C₂₋₆ alkynyl, C₁₋₆ haloalkyl, C₁₋₆ alkoxy(C₁₋₆)alkyl, C₁₋₆ alkoxy, C₃₋₆ cycloalkyl, nitro, cyano, C₁₋₆ haloalkoxy, S(O)ₚR¹¹, OSO₂R¹², NR¹³SO₂R¹⁴, NR¹⁵R¹⁶, NR¹⁷COR¹⁸, COR¹⁹, SiR²⁰R²¹R²², SCN, optionally substituted aryl or optionally substituted heteroaryl.

B is preferably O.

Het is preferably a heterocycle selected from heterocycles (a), (c), (f) and (g).

Het is more preferably a heterocycle selected from heterocycles (a), (c) and (g).

Het is even more preferably a heterocycle selected from heterocycles (a) and (c).

It is preferred that R¹ is hydrogen, C₁₋₂ alkyl or (C₁₋₆) alkoxymethyl.

It is more preferred that R¹ is hydrogen, ethyl, CH₂OCH₃ or CH₂OC₂H₅.

It is still more preferred that R¹ is hydrogen, ethyl or CH₂OC₂H₅.

It is even more preferred that R¹ is hydrogen or CH₂OC₂H₅.

It is preferred that R² is hydrogen or methyl.

In one aspect of the invention, it is preferred that R² is methyl.

It is preferred that R³, R⁴ and R⁵ are each, independently, hydrogen or halogen.

It is preferred that R³ is hydrogen or fluorine.

It is more preferred that R³ is hydrogen.

It is preferred that R⁴ is hydrogen or fluorine.

It is more preferred that R⁴ is hydrogen.

It is preferred that R⁵ is hydrogen or fluorine.

It is more preferred that R⁵ is hydrogen.

It is preferred that R⁷, R⁸ and R⁹ are each, independently, hydrogen, halogen, C₁₋₆ alkyl, C₁₋₆ haloalkyl, C₁₋₆ alkoxy, C₁₋₆ alkoxy(C₁₋₆)alkoxy, C₂₋₆ alkynyloxy, nitro, cyano, C₁₋₆ alkylthio, C₁₋₆ alkylsulfonyl or C₂₋₆ haloalkenyloxy.

It is preferred that R⁷ is hydrogen, halogen, C₁₋₆ alkyl, C₁₋₆ alkoxy(C₁₋₆)alkoxy, nitro or cyano.

It is more preferred that R⁷ is hydrogen, chlorine, fluorine, methyl, OC₂H₄OCH₃, nitro or cyano.

It is even more preferred that R⁷ is hydrogen or chlorine.

It is yet more preferred that R⁷ is hydrogen.

It is preferred that R⁸ is hydrogen, halogen, C₁₋₆ haloalkyl, C₁₋₆ alkoxy, C₁₋₆ alkoxy(C₁₋₆)alkoxy, C₂₋₆ alkynyloxy, cyano, C₁₋₆ alkylsulfonyl or C₂₋₆ haloalkenyloxy.

It is more preferred that R⁸ is hydrogen, chlorine, fluorine, bromine, CF₃, ethoxy, OC₂H₄OCH₃, OCH₂C.CH, cyano, SO₂CH₃ or OCH₂CH═CCl₂.

It is even more preferred that R⁸ is hydrogen, chlorine, CN, CF₃ or SO₂CH₃.

It is yet more preferred that R⁸ is hydrogen.

It is preferred that R⁹ is hydrogen, halogen or C₁₋₆ alkylthio.

It is more preferred that R⁹ is hydrogen, chlorine, fluorine, iodine or SCH₃.

It is even more preferred that R⁹ is hydrogen, chlorine or fluorine.

It is yet more preferred that R⁹ is hydrogen.

It is preferred that R⁶ and R¹⁰ are, independently, hydrogen, halogen, C₁₋₃ alkyl, C₁₋₂ haloalkyl, C₁₋₂ alkoxy, nitro, cyano, C₁₋₂ haloalkoxy, C₁₋₈ alkylthio or C₁₋₆ alkylsulfinyl, C₁₋₆ alkylsulfonyl; provided that at least one of R⁶ and R¹⁰ is not hydrogen.

In one aspect of the invention, it is preferred that R⁶ and R¹⁰ are, independently, hydrogen, halogen, C₁₋₃ alkyl, C₁₋₂ haloalkyl, C₁₋₂ alkoxy, nitro, cyano, C₁₋₂ haloalkoxy or C₁₋₂ alkylthio, provided that at least one of R⁶ and R¹⁰ is not hydrogen.

It is more preferred that R⁶ is hydrogen, methyl, chlorine, fluorine or bromine and R¹⁰ is hydrogen, methyl, chlorine, fluorine, OCH₃, SCH₃, CF₃ or nitro, provided that at least one of R⁶ and R¹⁰ is not hydrogen.

It is still more preferred that R⁶ is hydrogen, chlorine, fluorine or bromine and R¹⁰ is hydrogen, chlorine, fluorine, OCH₃, SCH₃, CF₃ or nitro, provided that at least one of R⁶ and R¹⁰ is not hydrogen.

It is even more preferred that R⁶ is hydrogen, chlorine, fluorine or bromine and R¹⁰ is chlorine, fluorine or bromine.

It is most preferred that when R⁶ is hydrogen, R¹⁰ is fluorine, chlorine or bromine and that when R⁶ is chlorine or fluorine, R¹⁰ is fluorine.

The compounds in Table 1 illustrate compounds of the invention. Table 1 provides 207 compounds of formula (I) where B is oxygen and R³, R⁴ and R⁵ are all H.

TABLE 1

| Comp. | | | | | | | | |
|---|---|---|---|---|---|---|---|---|
| No. | Het | R¹ | R² | R⁶ | R⁷ | R⁸ | R⁹ | R¹⁰ |
| 1 | (c) | H | CH₃ | Cl | H | H | H | F |
| 2 | (c) | H | CH₃ | F | H | H | H | F |
| 3 | (a) | H | CH₃ | Cl | H | H | H | OCH₃ |
| 4 | (c) | H | CH₃ | Cl | H | H | H | OCH₃ |
| 5 | (c) | H | CH₃ | Br | H | H | H | H |
| 6 | (c) | H | CH₃ | F | H | H | H | H |
| 7 | (c) | H | CH₃ | Cl | H | H | H | H |
| 8 | (a) | H | CH₃ | Cl | H | H | H | F |
| 9 | (c) | H | CH₃ | Cl | H | Cl | H | Cl |
| 10 | (c) | H | CH₃ | Cl | H | H | H | Cl |
| 11 | (c) | H | CH₃ | Cl | Cl | H | H | H |
| 12 | (c) | H | CH₃ | Cl | H | H | Cl | H |
| 13 | (c) | H | CH₃ | Cl | H | SO₂CH₃ | H | H |
| 14 | (c) | H | CH₃ | Cl | H | Cl | H | H |
| 15 | (a) | CH₂CH₃ | CH₃ | Cl | H | H | H | F |
| 16 | (c) | CH₂CH₃ | CH₃ | Cl | H | H | H | F |
| 17 | (c) | H | CH₃ | F | H | CF₃ | H | H |
| 18 | (c) | H | CH₃ | F | Cl | H | H | CF₃ |
| 19 | (c) | H | CH₃ | F | H | H | H | CF₃ |
| 20 | (c) | H | CH₃ | Cl | H | Cl | F | H |
| 21 | (c) | CH₂OC₂H₅ | CH₃ | Cl | H | H | H | F |
| 22 | (a) | CH₂OC₂H₅ | CH₃ | Cl | H | H | H | F |
| 23 | (c) | CH₂CH₃ | H | Cl | H | H | H | F |
| 24 | (g) | H | CH₃ | Cl | H | H | H | F |
| 25 | (c) | H | CH₃ | Cl | H | H | H | SCH₃ |
| 26 | (c) | H | CH₃ | H | H | H | H | SCH₃ |
| 27 | (g) | CH₂OC₂H₅ | CH₃ | Cl | H | H | H | F |
| 28 | (c) | CH₂OC₂H₅ | CH₃ | F | H | CF₃ | H | H |
| 29 | (c) | CH₂OC₂H₅ | CH₃ | Cl | H | Cl | F | H |
| 30 | (c) | H | CH₃ | H | H | H | H | NO₂ |
| 31 | (c) | H | CH₃ | H | H | H | H | OCH₃ |
| 32 | (c) | H | CH₃ | Cl | H | CN | H | H |
| 33 | (a) | H | CH₃ | CH₃ | CH₃ | H | H | H |
| 34 | (a) | H | CH₃ | F | H | H | H | F |
| 35 | (a) | H | CH₃ | NO₂ | H | H | H | H |
| 36 | (a) | H | CH₃ | Cl | H | Cl | H | H |
| 37 | (a) | H | CH₃ | Cl | H | H | H | H |
| 38 | (a) | H | CH₃ | NO₂ | H | Cl | H | H |
| 39 | (a) | H | CH₃ | Cl | H | F | H | H |
| 40 | (a) | H | CH₃ | F | H | Cl | H | H |
| 41 | (a) | H | CH₃ | Cl | H | Br | H | H |
| 42 | (a) | H | CH₃ | F | F | H | H | F |
| 43 | (a) | H | CH₃ | OCF₃ | H | H | H | H |
| 44 | (a) | H | CH₃ | F | F | F | H | H |
| 45 | (a) | H | CH₃ | F | F | F | H | F |
| 46 | (a) | H | CH₃ | SC₆H₁₃ | H | H | H | H |
| 47 | (a) | H | CH₃ | Cl | H | OC₂H₅ | H | H |
| 48 | (a) | H | CH₃ | Cl | H | OCH₂CH═CCl₂ | H | H |
| 49 | (a) | H | CH₃ | Cl | H | OC₂H₄OCH₃ | H | H |
| 50 | (a) | H | CH₃ | Cl | H | H | SCH₃ | H |
| 51 | (a) | H | CH₃ | SCH₃ | H | H | H | H |
| 52 | (a) | H | CH₃ | SOCH₃ | H | H | H | H |
| 53 | (a) | H | CH₃ | SO₂CH₃ | H | H | H | H |
| 54 | (a) | H | CH₃ | CN | H | H | H | H |
| 55 | (a) | H | CH₃ | Cl | H | OCH₂CCH | H | H |
| 56 | (a) | H | CH₃ | Cl | H | F | I | H |
| 57 | (a) | H | CH₃ | CH₃ | NO₂ | H | H | H |
| 58 | (a) | H | CH₃ | OCH₃ | CN | H | H | H |
| 59 | (a) | H | CH₃ | Cl | OC₂H₄OCH₃ | SO₂CH₃ | H | H |
| 60 | (a) | H | H | NO₂ | H | Cl | H | H |
| 61 | (a) | H | H | F | H | Cl | H | H |
| 62 | (a) | H | H | Cl | H | Br | H | H |
| 63 | (a) | H | H | OCF₃ | H | H | H | H |
| 64 | (a) | H | H | F | F | F | H | F |
| 65 | (a) | H | H | SC₆H₁₃ | H | H | H | H |
| 66 | (a) | H | H | Cl | H | OC₂H₅ | H | H |
| 67 | (a) | H | H | Cl | H | OCH₂CH═CCl₂ | H | H |
| 68 | (a) | H | H | Cl | H | OC₂H₄OCH₃ | H | H |
| 69 | (a) | H | H | Cl | H | H | SCH₃ | H |
| 70 | (a) | H | H | SCH₃ | H | H | H | H |
| 71 | (a) | H | H | SOCH₃ | H | H | H | H |
| 72 | (a) | H | H | SO₂CH₃ | H | H | H | H |
| 73 | (a) | H | H | Cl | H | OCH₂CCH | H | H |
| 74 | (a) | H | H | Cl | H | F | I | H |
| 75 | (a) | H | H | CH₃ | NO₂ | H | H | H |
| 76 | (a) | H | H | OCH₃ | CN | H | H | H |
| 77 | (a) | H | H | Cl | OC₂H₄OCH₃ | SO₂CH₃ | H | H |
| 78 | (a) | CH₂OC₂H₅ | H | NO₂ | H | Cl | H | H |
| 79 | (a) | CH₂OC₂H₅ | H | F | H | Cl | H | H |
| 80 | (a) | CH₂OC₂H₅ | H | Cl | H | Br | H | H |
| 81 | (a) | CH₂OC₂H₅ | H | OCF₃ | H | H | H | H |
| 82 | (a) | CH₂OC₂H₅ | H | F | F | F | H | F |
| 83 | (a) | CH₂OC₂H₅ | H | SC₆H₁₃ | H | H | H | H |
| 84 | (a) | CH₂OC₂H₅ | H | Cl | H | OC₂H₅ | H | H |
| 85 | (a) | CH₂OC₂H₅ | H | Cl | H | OCH₂CH═CCl₂ | H | H |
| 86 | (a) | CH₂OC₂H₅ | H | Cl | H | OC₂H₄OCH₃ | H | H |
| 87 | (a) | CH₂OC₂H₅ | H | Cl | H | H | SCH₃ | H |
| 88 | (a) | CH₂OC₂H₅ | H | SCH₃ | H | H | H | H |
| 89 | (a) | CH₂OC₂H₅ | H | SOCH₃ | H | H | H | H |
| 90 | (a) | CH₂OC₂H₅ | H | SO₂CH₃ | H | H | H | H |
| 91 | (a) | CH₂OC₂H₅ | H | Cl | H | OCH₂CCH | H | H |
| 92 | (a) | CH₂OC₂H₅ | H | Cl | H | F | I | H |
| 93 | (a) | CH₂OC₂H₅ | H | CH₃ | NO₂ | H | H | H |
| 94 | (a) | CH₂OC₂H₅ | H | OCH₃ | CN | H | H | H |
| 95 | (a) | CH₂OC₂H₅ | H | Cl | OC₂H₄OCH₃ | SO₂OH₃ | H | H |
| 96 | (a) | CH₂OC₂H₅ | CH₃ | CH₃ | CH₃ | H | H | H |
| 97 | (a) | CH₂OC₂H₅ | CH₃ | F | H | H | H | F |
| 98 | (a) | CH₂OC₂H₅ | CH₃ | NO₂ | H | H | H | H |
| 99 | (a) | CH₂OC₂H₅ | CH₃ | Cl | H | Cl | H | H |
| 100 | (a) | CH₂OC₂H₅ | CH₃ | Cl | H | H | H | H |
| 101 | (a) | CH₂OC₂H₅ | CH₃ | NO₂ | H | Cl | H | H |
| 102 | (a) | CH₂OC₂H₅ | CH₃ | Cl | H | F | H | H |
| 103 | (a) | CH₂OC₂H₅ | CH₃ | F | H | Cl | H | H |
| 104 | (a) | CH₂OC₂H₅ | CH₃ | Cl | H | Br | H | H |
| 105 | (a) | CH₂OC₂H₅ | CH₃ | F | F | H | H | F |
| 106 | (a) | CH₂OC₂H₅ | CH₃ | OCF₃ | H | H | H | H |
| 107 | (a) | CH₂OC₂H₅ | CH₃ | F | F | F | H | H |
| 108 | (a) | CH₂OC₂H₅ | CH₃ | F | F | F | H | F |
| 109 | (a) | CH₂OC₂H₅ | CH₃ | SC₆H₁₃ | H | H | H | H |
| 110 | (a) | CH₂OC₂H₅ | CH₃ | Cl | H | OC₂H₅ | H | H |
| 111 | (a) | CH₂OC₂H₅ | CH₃ | Cl | H | OCH₂CH═CCl₂ | H | H |
| 112 | (a) | CH₂OC₂H₅ | CH₃ | Cl | H | OC₂H₄OCH₃ | H | H |
| 113 | (a) | CH₂OC₂H₅ | CH₃ | Cl | H | H | SCH₃ | H |
| 114 | (a) | CH₂OC₂H₅ | CH₃ | SCH₃ | H | H | H | H |
| 115 | (a) | CH₂OC₂H₅ | CH₃ | SOCH₃ | H | H | H | H |
| 116 | (a) | CH₂OC₂H₅ | CH₃ | SO₂CH₃ | H | H | H | H |
| 117 | (a) | CH₂OC₂H₅ | CH₃ | CN | H | H | H | H |
| 118 | (a) | CH₂OC₂H₅ | CH₃ | Cl | H | OCH₂CCH | H | H |
| 119 | (a) | CH₂OC₂H₅ | CH₃ | Cl | H | F | I | H |
| 120 | (a) | CH₂OC₂H₅ | CH₃ | CH₃ | NO₂ | H | H | H |
| 121 | (a) | CH₂OC₂H₅ | CH₃ | OCH₃ | CN | H | H | H |
| 122 | (a) | CH₂OC₂H₅ | CH₃ | Cl | OC₂H₄OCH₃ | SO₂CH₃ | H | H |
| 123 | (c) | H | CH₃ | CH₃ | CH₃ | H | H | H |
| 124 | (c) | H | CH₃ | NO₂ | H | Cl | H | H |
| 125 | (c) | H | CH₃ | Cl | H | F | H | H |
| 126 | (c) | H | CH₃ | F | H | Cl | H | H |
| 127 | (c) | H | CH₃ | Cl | H | Br | H | H |
| 128 | (c) | H | CH₃ | F | F | H | H | F |
| 129 | (c) | H | CH₃ | OCF₃ | H | H | H | H |
| 130 | (c) | H | CH₃ | F | F | F | H | H |
| 131 | (c) | H | CH₃ | F | F | F | H | F |
| 132 | (c) | H | CH₃ | SC₆H₁₃ | H | H | H | H |
| 133 | (c) | H | CH₃ | Cl | H | OC₂H₅ | H | H |
| 134 | (c) | H | CH₃ | Cl | H | OCH₂CH═CCl₂ | H | H |
| 135 | (c) | H | CH₃ | Cl | H | OC₂H₄OCH₃ | H | H |
| 136 | (c) | H | CH₃ | CL | H | H | SCH₃ | H |
| 137 | (c) | H | CH₃ | SOCH₃ | H | H | H | H |
| 138 | (c) | H | CH₃ | SO₂CH₃ | H | H | H | H |
| 139 | (c) | H | CH₃ | CN | H | H | H | H |
| 140 | (c) | H | CH₃ | Cl | H | OCH₂CCH | H | H |
| 141 | (c) | H | CH₃ | Cl | H | F | I | H |
| 142 | (c) | H | CH₃ | CH₃ | NO₂ | H | H | H |
| 143 | (c) | H | CH₃ | OCH₃ | CN | H | H | H |
| 144 | (c) | H | CH₃ | Cl | OC₂H₄OCH₃ | SO₂CH₃ | H | H |
| 145 | (c) | H | H | NO₂ | H | Cl | H | H |
| 146 | (c) | H | H | F | H | Cl | H | H |
| 147 | (c) | H | H | Cl | H | Br | H | H |
| 148 | (c) | H | H | OCF₃ | H | H | H | H |
| 149 | (c) | H | H | F | F | F | H | F |
| 150 | (c) | H | H | SC₆H₁₃ | H | H | H | H |
| 151 | (c) | H | H | Cl | H | OC₂H₅ | H | H |
| 152 | (c) | H | H | Cl | H | OCH₂CH═CCl₂ | H | H |
| 153 | (c) | H | H | Cl | H | OC₂H₄OCH₃ | H | H |
| 154 | (c) | H | H | Cl | H | H | SCH₃ | H |
| 155 | (c) | H | H | SCH₃ | H | H | H | H |
| 156 | (c) | H | H | SOCH₃ | H | H | H | H |
| 157 | (c) | H | H | SO₂CH₃ | H | H | H | H |
| 158 | (c) | H | H | Cl | H | OCH₂CCH | H | H |
| 159 | (c) | H | H | Cl | H | F | I | H |
| 160 | (c) | H | H | CH₃ | NO₂ | H | H | H |
| 161 | (c) | H | H | OCH₃ | CN | H | H | H |
| 162 | (c) | H | H | Cl | OC₂H₄OCH₃ | SO₂CH₃ | H | H |
| 163 | (c) | CH₂OC₂H₅ | H | NO₂ | H | Cl | H | H |
| 164 | (c) | CH₂OC₂H₅ | H | F | H | Cl | H | H |
| 165 | (c) | CH₂OC₂H₅ | H | Cl | H | Br | H | H |
| 166 | (c) | CH₂OC₂H₅ | H | OCF₃ | H | H | H | H |
| 167 | (c) | CH₂OC₂H₅ | H | F | F | F | H | F |
| 168 | (c) | CH₂OC₂H₅ | H | SC₆H₁₃ | H | H | H | H |
| 169 | (c) | CH₂OC₂H₅ | H | Cl | H | OC₂H₅ | H | H |
| 170 | (c) | CH₂OC₂H₅ | H | Cl | H | OCH₂CH═CCl₂ | H | H |
| 171 | (c) | CH₂OC₂H₅ | H | Cl | H | OC₂H₄OCH₃ | H | H |
| 172 | (c) | CH₂OC₂H₅ | H | Cl | H | H | SCH₃ | H |
| 173 | (c) | CH₂OC₂H₅ | H | SCH₃ | H | H | H | H |
| 174 | (c) | CH₂OC₂H₅ | H | SOCH₃ | H | H | H | H |
| 175 | (c) | CH₂OC₂H₅ | H | SO₂CH₃ | H | H | H | H |
| 176 | (c) | CH₂OC₂H₅ | H | Cl | H | OCH₂CCH | H | H |
| 177 | (c) | CH₂OC₂H₅ | H | Cl | H | F | I | H |
| 178 | (c) | CH₂OC₂H₅ | H | CH₃ | NO₂ | H | H | H |
| 179 | (c) | CH₂OC₂H₅ | H | OCH₃ | CN | H | H | H |
| 180 | (c) | CH₂OC₂H₅ | H | Cl | OC₂H₄OCH₃ | SO₂CH₃ | H | H |
| 181 | (c) | CH₂OC₂H₅ | CH₃ | CH₃ | CH₃ | H | H | H |
| 182 | (c) | CH₂OC₂H₅ | CH₃ | F | H | H | H | F |
| 183 | (c) | CH₂OC₂H₅ | CH₃ | NO₂ | H | H | H | H |
| 184 | (c) | CH₂OC₂H₅ | CH₃ | Cl | H | Cl | H | H |
| 185 | (c) | CH₂OC₂H₅ | CH₃ | Cl | H | H | H | H |
| 186 | (c) | CH₂OC₂H₅ | CH₃ | NO₂ | H | Cl | H | H |
| 187 | (c) | CH₂OC₂H₅ | CH₃ | Cl | H | F | H | H |
| 188 | (c) | CH₂OC₂H₅ | CH₃ | F | H | Cl | H | H |
| 189 | (c) | CH₂OC₂H₅ | CH₃ | Cl | H | Br | H | H |
| 190 | (c) | CH₂OC₂H₅ | CH₃ | F | F | H | H | F |
| 191 | (c) | CH₂OC₂H₅ | CH₃ | OCF₃ | H | H | H | H |
| 192 | (c) | CH₂OC₂H₅ | CH₃ | F | F | F | H | H |
| 193 | (c) | CH₂OC₂H₅ | CH₃ | F | F | F | H | F |
| 194 | (c) | CH₂OC₂H₅ | CH₃ | SC₆H₁₃ | H | H | H | H |
| 195 | (c) | CH₂OC₂H₅ | CH₃ | Cl | H | OC₂H₅ | H | H |
| 196 | (c) | CH₂OC₂H₅ | CH₃ | Cl | H | OCH₂CH═CCl₂ | H | H |
| 197 | (c) | CH₂OC₂H₅ | CH₃ | Cl | H | OC₂H₄OCH₃ | H | H |
| 198 | (c) | CH₂OC₂H₅ | CH₃ | Cl | H | H | SCH₃ | H |
| 199 | (c) | CH₂OC₂H₅ | CH₃ | SCH₃ | H | H | H | H |
| 200 | (c) | CH₂OC₂H₅ | CH₃ | SOCH₃ | H | H | H | H |
| 201 | (c) | CH₂OC₂H₅ | CH₃ | SO₂CH₃ | H | H | H | H |
| 202 | (c) | CH₂OC₂H₅ | CH₃ | CN | H | H | H | H |
| 203 | (c) | CH₂OC₂H₅ | CH₃ | Cl | H | OCH₂CCH | H | H |
| 204 | (c) | CH₂OC₂H₅ | CH₃ | Cl | H | F | I | H |
| 205 | (c) | CH₂OC₂H₅ | CH₃ | CH₃ | NO₂ | H | H | H |
| 206 | (c) | CH₂OC₂H₅ | CH₃ | OCH₃ | CN | H | H | H |
| 207 | (c) | CH₂OC₂H₅ | CH₃ | Cl | OC₂H₄OCH₃ | SO₂CH₃ | H | H |

The following abbreviations are used throughout this description:

| | |
|---|---|
| m.p. = melting point | ppm = parts per million |
| s = singlet | br = broad |
| d = doublet | dd = doublet of doublets |
| t = triplet | q = quartet |
| m = multiplet | |

Table 2 shows selected melting point and selected NMR data, all with CDCl₃ as the solvent (unless otherwise stated; if a mixture of solvents is present, this is indicated as, for example, (CDCl₃/d₆-DMSO)), (no attempt is made to list all characterising data in all cases) for compounds of formula (I).

TABLE 2

| Compound | M.P. | NMR proton shifts (/ppm) |
|---|---|---|
| No. | (/° C.) | (CDCl₃ unless otherwise stated) |
| 1 | 178-179 | 1.27(t, 3H); 1.71(d, 3H); 2.71(q, 2H); 4.08(q, 1H); 7.2(t, 1H); |
| | | 7.39(d, 1H); 7.48(m, 2H); 7.68(d, 1H); 7.88(d, 1H); |
| | | 8.3(br, 1H). |
| 2 | 214-216 | 1.26(t, 3H); 1.72(d, 3H); 2.72(q, 2H); 4.03(m, 1H); |
| | | 7.13(m, 2H); 7.42(dd, 1H); 752(m, 1H); 7.69(d, 1H); |
| | | 7.87(s, 1H); 8.02(br, 1H). |
| 3 | 211-212 | 1.71(d, 3H); 2.38(s, 3H); 3.8(s, 3H); 4.05(m, 1H); |
| | | 6.92(d, 1H); 7.12(d, 1H); 7.42(m, 2H); 7.65(d, 1H); |
| | | 7.82(d, 1H); 8.18(br, 1H). |
| 4 | 178-179 | 1.27(t, 3H); 1.72(d, 3H); 2.72(q, 2H); 3.81(s, 3H); |
| | | 4.07(q, 1H); 6.94(d, 1H); 7.13(d, 1H); 7.42(m, 2H); |
| | | 7.64(d, 1H); 7.82(d, 1H); 8.28(br, 1H) |
| 5 | 117-118 | 1.26(t, 3H); 1.73(d, 3H); 2.73(q, 2H); 4.06(q, 1H); |
| | | 7.34-7.52(m, 3H); 7.66(d, 1H); 7.78(d, 1H); 7.84(s, 1H); |
| | | 8.09(d, 1H); 8.23(br, 1H) |
| 6 | 200-202 | 1.27(t, 3H); 1.74(d, 3H); 2.72(q, 2H); 4.05(q, 1H); |
| | | 7.27-7.44(m, 3H); 7.51-7.60(m, 1H); 7.66(d, 1H); |
| | | 7.83(s, 1H); 8.06(br, 1H); 8.25(m, 1H) |
| 7 | | 1.26(t, 3H); 1.74(d, 3H); 2.73(q, 2H); 4.06(q, 1H); |
| | | 7.36-7.54(m, 3H); 7.59(d, 1H); 7.66(d, 1H); 7.86(s, 1H); |
| | | 8.09(br, 1H); 8.16(d, 1H) |
| 8 | 175-176 | 1.72(d, 3H); 2.38(s, 3H); 4.06(q, 1H); 7.2(t, 1H); |
| | | 7.45(m, 3H); 7.69(d, 1H); 7.89(d, 1H); 8.13(br, 1H) |
| 9 | 69-73 | 1.27(t, 3H); 1.76(d, 3H); 2.74(q, 2H); 4.07(q, 1H); |
| | | 7.48(d, 1H); 7.53(s, 2H); 7.68(d, 1H); 7.89(s, 1H); |
| | | 8.11(s, 1H) |
| 10 | 65-70 | 1.27(t, 3H); 1.74(d, 3H); 2.74(q, 2H); 4.07(q, 1H); |
| | | 7.33-7.39(m, 1H); 7.43-7.52(m, 3H); 7.67(d, 1H); |
| | | 7.89(s, 1H); 8.16(s, 1H) |
| 11 | 158-161 | 1.27(t, 3H); 1.75(d, 3H); 2.73(q, 2H); 4.06(q, 1H); |
| | | 7.35-7.46(m, 2H); 7.64-7.73(m, 2H); 7.85(s, 1H); |
| | | 7.99-8.10(m, 2H) |
| 12 | 177-179 | 1.26(t, 3H); 1.75(d, 3H); 2.74(q, 2H); 4.05(q, 1H); |
| | | 7.41-7.48(m, 2H); 7.54(d, 1H); 7.67(d, 1H); 7.86(s, 1H); |
| | | 8.04(s, 1H); 8.19(s, 1H) |
| 13 | 92-96 | 1.27(t, 3H); 1.75(d, 3H); 2.74(q, 2H); 3.15(s, 3H); |
| | | 4.06(q, 1H); 7.46(d, 1H); 7.72(d, 1H); 7.89(s, 1H); |
| | | 7.99(d, 1H); 8.06(s, 1H); 8.17(s, 1H); 8.43(d, 1H) |
| 14 | 153-156 | 1.27(t, 3H); 1.74(d, 3H); 2.74(q, 2H); 4.06(q, 1H); |
| | | 7.38-7.46(m, 2H); 7.61(s, 1H); 7.67(d, 1H); 7.85(s, 1H); |
| | | 8.05(s, 1H); 8.15(d, 1H) |
| 15 | gum | 1.14(t, 3H); 1.52(d, 3H); 3.6(br, 1H); 3.85(br, 2H); |
| | | 7.16(br, 2H); 7.37(dd, 1H); 7.5(m, 3H) |
| 16 | gum | 1.14(t, 3H); 1.32(t, 3H); 1.51(d, 3H); 2.8(q, 2H); 3.6(br, 1H); |
| | | 3.89(br, 2H); 7.14(m, 2H); 7.38(d, 1H); 7.49(m, 3H) |
| 17 | 158-161 | 1.26(t, 3H); 1.74(d, 3H); 2.73(q, 2H); 4.06(q, 1H); |
| | | 7.45(d, 1H); 7.54-7.65(m, 2H); 7.69(d, 1H); 7.87(s, 1H); |
| | | 8.06(s, 1H); 8.41(t, 1H) |
| 18 | 55-59 | 1.23-1.32(m, 3H); 1.74(d, 3H); 2.74(q, 2H); 4.07(q, 1H), |
| | | 7.49(d, 1H); 7.64(d, 1H); 7.69(d, 1H); 7.78(t, 1H); |
| | | 7.89(s, 1H); 8.07(s, 1H) |
| 19 | 153-156 | 1.27(t, 3H); 1.74(d, 3H); 2.74(q, 2H); 4.06(q, 1H); |
| | | 7.40-7.55(m, 2H); 7.62-7.76(m, 3H); 7.87(s, 1H); 8.09(s, 1H) |
| 20 | 171-174 | 1.28(t, 3H); 1.74(d, 3H); 2.73(q, 2H); 4.06(q, 1H); |
| | | 7.45(d, 1H); 7.63-7.72(m, 2H); 7.86(s, 1H); 8.03(d, 1H); |
| | | 8.06(s, 1H) |
| 21 | gum | 1.15(t, 3H); 1.32(t, 3H); 1.53(d, 3H); 2.8(br, 2H); |
| | | 3.55(br, 2H); 3.9(br, 1H); 5.1(br, 2H); 7.15(br, 1H); |
| | | 7.18(t, 1H); 7.39(d, 1H); 7.5(m, 3H) |
| 22 | gum | 1.15(t, 3H); 1.52(d, 3H); 2.5(s, 3H); 2.53(m, 2H); 3.9(br, 1H); |
| | | 5.1(br, 2H); 7.13(br, 1H); 7.19(t, 1H); 7.39(d, 1H); |
| | | 7.52(m, 3H) |
| 23 | gum | 1.2(t, 3H); 1.35(t, 3H); 2.84(q, 2H); 3.7(br, 2H); 3.79(br, 2H); |
| | | 7.19(m, 2H); 7.38(d, 1H); 7.48(m, 1H); 7.55(m, 2H) |
| 24 | gum | 1.78(d, 3H); 4.1(q, 1H); 7.01(m, 1H); 7.2(t, 1H) 7.35(m, 3H); |
| | | 7.51(m, 2H); 7.72(d, 1H); 7.89(br, 1H); 7.98(s, 1H); |
| | | 8.34(d, 1H); 8.88(d, 1H) |
| 25 | gum | 1.38(t, 3H); 1.74(d, 3H); 2.45(s, 3H); 2.73(q, 2H); |
| | | 4.05(q, 1H); 7.3(m, 2H); 7.43(m, 2H); 7.68(d, 1H); |
| | | 7.88(s, 1H); 8.08(br, 1H) |
| 26 | 140-141 | 1.26(t, 3H); 1.73(d, 3H); 2.57(s, 3H); 2.72(q, 2H); |
| | | 4.03(m, 1H); 7.28(m, 1H); 7.37(m, 2H); 7.63(d, 1H); |
| | | 7.89(s, 1H); 8.01(br, 1H); 8.2(dd, 1H) |
| 27 | gum | |
| 28 | gum | 1.15(t, 3H); 1.35(t, 3H); 1.54(d, 3H); 2.75-2.94(m, 2H); |
| | | 3.44-3.67(m, 2H); 3.80-4.00(m, 1H); 4.65-5.50(m, 2H); |
| | | 7.03-7.19(m, 1H); 7.44-7.64(m, 4H); 8.37(t, 1H) |
| 29 | gum | 1.18(t, 3H); 1.27(t, 3H); 1.74(d, 3H); 2.96(q, 2H); |
| | | 3.54(q, 2H); 4.39(q, 1H); 5.23(s, 2H); 7.49-7.58(m, 2H); |
| | | 7.64(d, 1H); 7.94(s, 1H); 7.97(d, 1H) |
| 30 | 183-184 | 1.26(t, 3H); 1.72(d, 3H); 2.72(q, 2H); 4.03(m, 1H); |
| | | 7.41(dd, 1H); 7.6(d, 1H); 7.74(m, 3H); 7.8(s, 1H); |
| | | 7.91(dd, 1H); 8.09(br, 1H); 8.13(d, 1H) |
| 31 | 220-222 | 1.26(t, 3H); 1.72(d, 3H); 2.7(q, 2H); 4.03(m, 4H); |
| | | 7.12(m, 2H); 7.32(d, 1H); 7.52(m, 1H); 7.62(d, 1H); |
| | | 7.8(s, 1H) 8.06(br, 1H); 8.14(d, 1H) |
| 32 | 179-180 | 1.25(t, 3H); 1.72(d, 3H); 2.71(q, 2H); 4.05(q, 1H); |
| | | 7.46(dd, 1H); 7.59(m, 2H); 7.88(m, 2H); 8.07(br, 1H); |
| | | 8.32(d, 1H) |
| 159 | 248-249 | |
| 160 | 204-206 | |
| 161 | 238-240 | |
| 162 | 158-159 | |

The compounds of the invention may be made in a variety of ways.

For example, a compound of formula (I) which is a compound of formula (A) (where Het, B, R¹, R², R³, R⁴, R⁵, R⁶, R⁷, R⁸, R⁹ and R¹⁰ are as defined above for a compound of formula (I) except that R¹ is not H) may be made from a compound of formula (I) which is a compound of formula (I) (where Het, B, R², R³, R⁴, R⁵, R⁶, R⁷, R⁸, R⁹ and R¹⁰ are as defined above for a compound of formula (I)) by treatment with an alkylating agent (such as an alkyl halide, dialkyl sulfate or trialkyloxonium salt) optionally in the presence of a base.

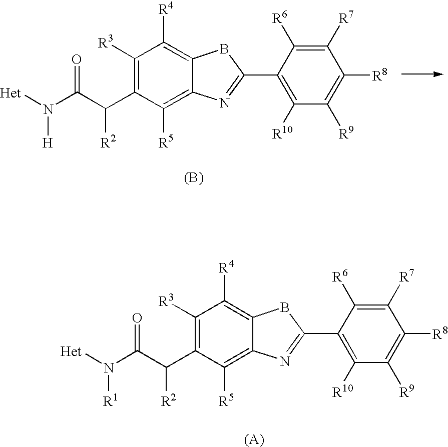

A compound of formula (I) which is a compound of formula (B) (where Het, B, R², R³, R⁴, R⁵, R⁶, R⁷, R⁸, R⁹ and R¹⁰ are as defined above for a compound of formula (I)) may be made from a compound of formula (C) by reacting a compound of formula (II) (where Het is as defined above for a compound of formula (I)) either with an appropriate compound of formula (C) (where B, R², R³, R⁴, R⁵, R⁶, R⁷, R⁸, R⁹ and R¹⁰ are as defined above for a compound of formula (I) and X is OH) preferably in the presence of a suitable coupling reagent (such as 1,3-dicyclohexylcarbodiimide, 1,3-diisopropylcarbodiimide, 1-(3-dimethylaminopropyl)-3-ethylcarbodiimide or 1,1′-carbonyldiimidazole) and optionally in the presence of a suitable additive (such as 1-hydroxybenzotriazole or 1-hydroxy-7-azabenzotriazole) in a suitable solvent (such as N,N-dimethylformamide) or with a suitable compound of formula (C) (where B, R², R³, R⁴, R⁵, R⁶, R⁷, R⁸, R⁹ and R¹⁰ are as defined above for a compound of formula (I) and X is halogen, acyloxy, alkoxy (especially methoxy), substituted alkoxy or aryloxy) optionally in the presence of a base (such as triethylamine or sodium methoxide) and in a suitable solvent (such as 1,1,2,2-tetrachloroethane, tetrahydrofuran, N,N-dimethylacetamide or mesitylene).

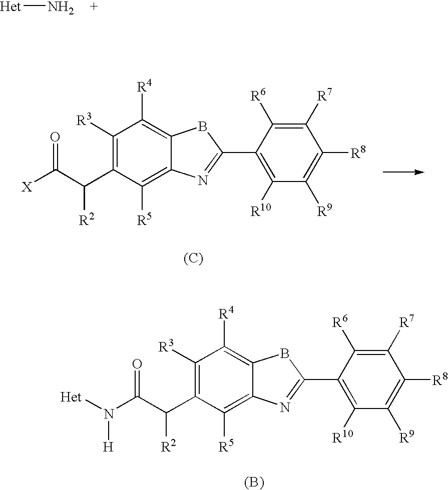

Compounds (II) are known compounds or can be made from known compounds by known methods.

A compound of formula (C) (where B, R², R³, R⁴, R⁵, R⁶, R⁷, R⁸, R⁹ and R¹⁰ are as defined above for a compound of formula (I) and X is hydroxy, halogen or acyloxy) may be prepared from a compound of formula (C) (where B, R², R³, R⁴, R⁵, R⁶, R⁷, R⁸, R⁹ and R¹⁰ are as defined above for a compound of formula (I) and X is alkoxy) by known methods.

A compound of formula (C) (where B, R³, R⁴, R⁵, R⁶, R⁷, R⁸, R⁹ and R¹⁰ are as defined above for a compound of formula (I), R² is methyl and X is alkoxy) may be prepared by treatment of a compound of formula (D) (where B, R³, R⁴, R⁵, R⁶, R⁷, R⁸, R⁹ and R¹⁰ are as defined above for a compound of formula (I) and X is alkoxy) with a suitable base (such as lithium diisopropylamide, sodium hydride or lithium bis(trimethylsilyl)amide) in a suitable solvent (such as tetrahydrofuran) and then treated with a suitable methylating reagent (for example methyl iodide or dimethyl sulfate).

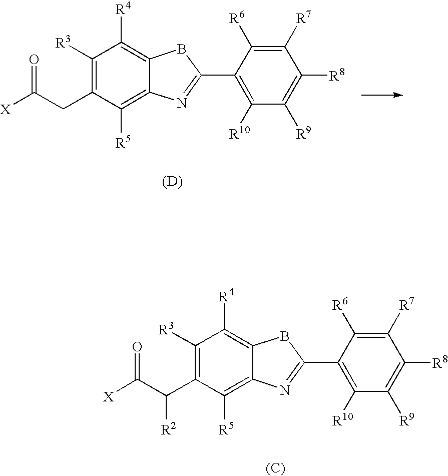

A compound of formula (C) (where B, R³, R⁴, R⁵, R⁶, R⁷, R⁸, R⁹ and R¹⁰ are as defined above for a compound of formula (I), R² is fluoro and X is alkoxy) may be prepared by treatment of a compound of formula (I)) (where B, R³, R⁴, R⁵, R⁶, R⁷, R⁸, R⁹ and R¹⁰ are as defined above for a compound of formula (I) and X is alkoxy) with a suitable base (such as lithium diisopropylamide, sodium hydride or lithium bis(trimethylsilyl)amide) in a suitable solvent (such as tetrahydrofuran) and then treated with a fluorinating agent (for example N-fluorobenzenesulfonimide).

A compound of formula (I)) (where B, R³, R⁴, R⁵, R⁶, R⁷, R⁸, R⁹ and R¹⁰ are as defined above for a compound of formula (I) and X is alkoxy) may be prepared from a compound of formula (E) (where B, R³, R⁴, and R⁵, are as defined above for a compound of formula (I) and X is alkoxy) under known conditions. For example, a compound of formula (E) (where B, R³, R⁴, and R⁵ are as defined above for a compound of formula (I) and X is alkoxy) may be acylated with a compound of formula (III) (where R⁶, R⁷, R⁸, R⁹ and R¹⁰ are as defined above for a compound of formula (I) and Y is chloro), optionally in the presence of a base (such as pyridine or sodium bicarbonate), and in a suitable solvent (such as N,N-dimethylacetamide or 1,2-dimethoxyethane) and cyclised (preferably in the presence of an acid such as para-toluenesulfonic acid or pyridinium para-toluenesulfonate) in a suitable solvent (such as toluene, xylene or 1,1,2,2-tetrachloroethane). Alternatively a compound of formula (E)(where B, R³, R⁴, and R⁵ are as defined above for a compound of formula (I) and X is alkoxy) may be acylated with a compound of formula (III) (where R⁶, R⁷, R⁸, R⁹ and R¹⁰ are as defined above for a compound of formula (I) and Y is OH) optionally in the presence of a suitable coupling reagent (such as 1,3-dicyclohexylcarbodiimide, 1,3-diisopropylcarbodiimide, 1-(3-dimethylaminopropyl)-3-ethylcarbodiimide or 1,1′-carbonyldiimidazole) and optionally in the presence of a suitable additive (such as 1-hydroxybenzotriazole or 1-hydroxy-7-azabenzotriazole) in a suitable solvent (such as N,N-dimethylacetamide) and cyclised (preferably in the presence of an acid such as para-toluenesulfonic acid or pyridinium para-toluenesulfonate) in a suitable solvent (such as xylene or 1,1,2,2-tetrachloroethane).

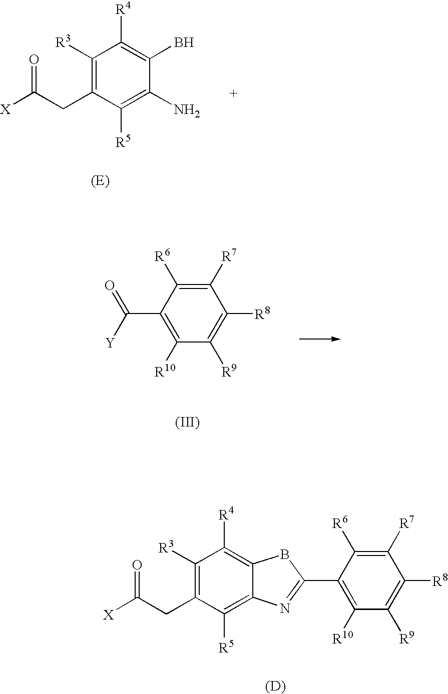

A compound of formula (E) (where B, R³, R⁴, and R⁵, are as defined above for a compound of formula (I) and X is alkoxy) may be prepared from a compound of formula (F) (where B, R³, R⁴, and R⁵, are as defined above for a compound of formula (I) and X is alkoxy) by reduction using procedures known in the art (see, for example, J. March, Advanced Organic Chemistry, Third Edition, John Wiley and Sons, New York, 1985, and references therein).

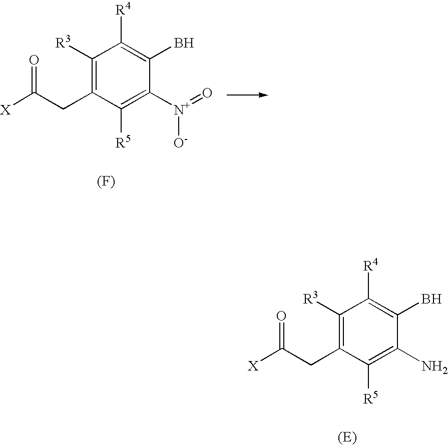

A compound of formula (F) (where R³, R⁴, and R⁵, are as defined above for a compound of formula (I), B is sulfur and X is alkoxy) may be prepared from a compound of formula (P) (where R³, R⁴, and R¹ are as defined above for a compound of formula (I), B is oxygen and X is alkoxy) using conditions similar to those described by J. Scheigetz, R. Zamboni and B. Roy, Synth. Commun., 25 (1995) (18), pages 2791-2806.

A compound of formula (P) (where R³, R⁴, and R⁵ are as defined above for a compound of formula (I), B is oxygen and X is alkoxy) may be prepared from a compound of formula (G) (where R³, R⁴, and R⁵ are as defined above for a compound of formula (I), B is oxygen and X is alkoxy) by nitration under known conditions.

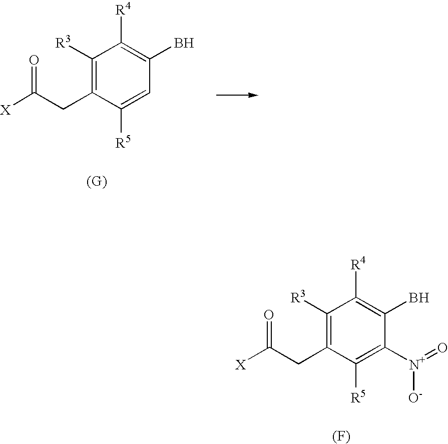

Compounds (G) are known compounds or may be made from known compounds by known methods.

Alternatively, a compound of formula (I) which is a compound of formula (B) (where Het, R², R³, R⁴, R⁵, R⁶, R⁷, R⁸, R⁹ and R¹⁰ are as defined above for a compound of formula (I) and B is oxygen) may be made from a compound of formula (H) (where Het, R², R³, R⁴, and R⁵ are as defined above for a compound of formula (I) and B is oxygen) under conditions similar to those described above.

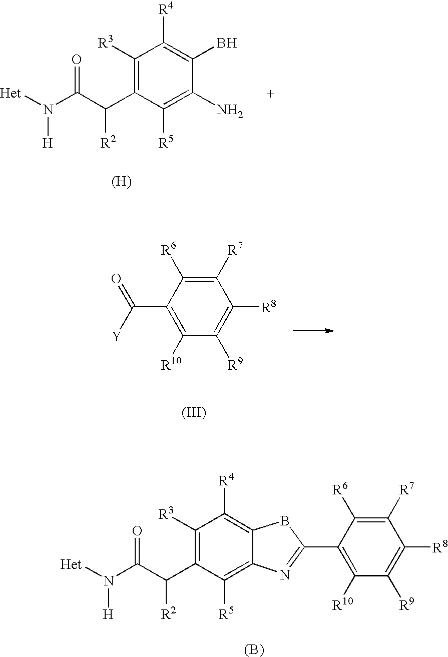

For example, a compound of formula (H) (where Het, R², R³, R⁴, and R⁵ are as defined above for a compound of formula (I) and B is oxygen) may be acylated with a compound of formula (III) (where R⁶, R⁷, R⁸, R⁹ and R¹⁰ are as defined above for a compound of formula (I) and Y is chloro), optionally in the presence of a base (such as pyridine), and in a suitable solvent (such as N,N-dimethylacetamide) and cyclised (preferably in the presence of an acid such as para-toluenesulfonic acid or pyridinium para-toluenesulfonate) in a suitable solvent (such as xylene or 1,1,2,2-tetrachloroethane).

Alternatively a compound of formula (H) (where Het, R², R³, R⁴, and R⁵ are as defined above for a compound of formula (I) and B is oxygen) may be acylated with a compound of formula (E) (where R⁶, R⁷, R⁸, R⁹ and R¹⁰ are as defined above for a compound of formula (I) and Y is OH) optionally in the presence of a suitable coupling reagent (such as 1,3-dicyclohexylcarbodiimide, 1,3-diisopropylcarbodiimide, 1-(3-dimethylaminopropyl)-3-ethylcarbodiimide or 1,1′-carbonyldiimidazole) and optionally in the presence of a suitable additive (such as 1-hydroxybenzotriazole or 1-hydroxy-7-azabenzotriazole) in a suitable solvent (such as N,N-dimethylacetamide) and cyclised (preferably in the presence of an acid such as para-toluenesulfonic acid or pyridinium para-toluenesulfonate) in a suitable solvent (such as xylene or 1,1,2,2-tetrachloroethane).

A compound of formula (M) (where R⁶, R⁷, R⁸, R⁹ and R¹⁰ are as defined above for a compound of formula (I) and Y is OH), may be treated according to the procedure described by Paul Froyen (Tetrahedron Letters, Vol. 38, No. 30, pp 5359-5362, 1997) and the resulting acyloxyphosphonium salt reacted with a compound of formula (H) (where Het, R², R³, R⁴ and R⁵ are as defined above for a compound of formula (I) and B is oxygen) and subsequently cyclised according to the procedures described above to give further compounds of formula (I) which are compounds of formula (B).

A compound of formula (H) (where Het, R², R³, R⁴ and R⁵ are as defined above for a compound of formula (I) and B is oxygen) may be prepared from a compound of formula (3) (where Het, R², R³, R⁴ and R⁵ are as defined above for a compound of formula (I) and B is oxygen) by reduction using procedures known in the art (see, for example, J. March, Advanced Organic Chemistry, Third Edition, John Wiley and Sons, New York, 1985, and references therein).

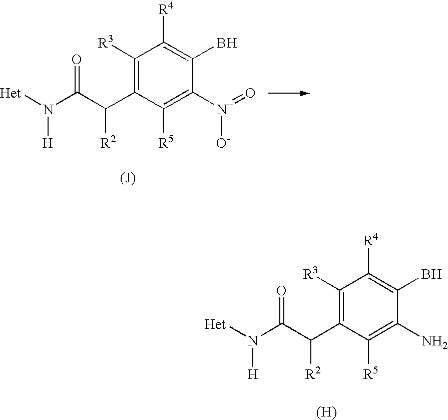

A compound of formula (J) (where Het, R², R³, R⁴ and R⁵ are as defined above for a compound of formula (I) and B is oxygen) may be prepared by treating a compound of formula (K) (where R², R³, R⁴ and R⁵ are as defined above for a compound of formula (I), B is oxygen and X is OH) with a compound of formula (II) (where Het is as defined above for a compound of formula (I)) preferably in the presence of a suitable coupling reagent (such as 1,3-dicyclohexylcarbodiimide, 1,3-diisopropylcarbodiimide, 1-(3-dimethylaminopropyl)-3-ethylcarbodiimide or 1,1′-carbonyldiimidazole) and optionally in the presence of a suitable additive (such as 1-hydroxybenzotriazole or 1-hydroxy-7-azabenzotriazole) in a suitable solvent (such as acetonitrile or N,N-dimethylacetamide).

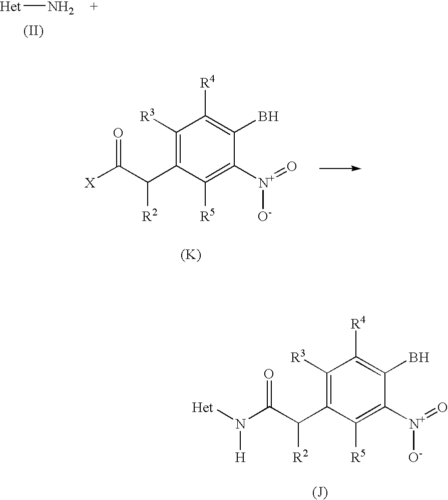

Alternatively, a compound of formula (J) (where Het, R², R³, R⁴ and R⁵ are as defined above for a compound of formula (I) and B is oxygen) may be prepared by treating a compound of formula (K) (where R², R³, R⁴ and R⁵ are as defined above for a compound of formula (I), B is oxygen and X is halogen, acyloxy, alkoxy (especially methoxy), substituted alkoxy or aryloxy) optionally in the presence of a base (such as triethylamine or sodium methoxide) and in a suitable solvent (such as 1,1,2,2-tetrachloroethane, tetrahydrofuran, N,N-dimethylacetamide or mesitylene).

A compound of formula (K) (where R², R³, R⁴ and R⁵, are as defined above for a compound of formula (I), B is oxygen and X is alkoxy) may be prepared from a compound of formula (L) (where R², R³, R⁴ and R⁵, are as defined above for a compound of formula (I), B is oxygen and X is alkoxy) by nitration under known conditions.

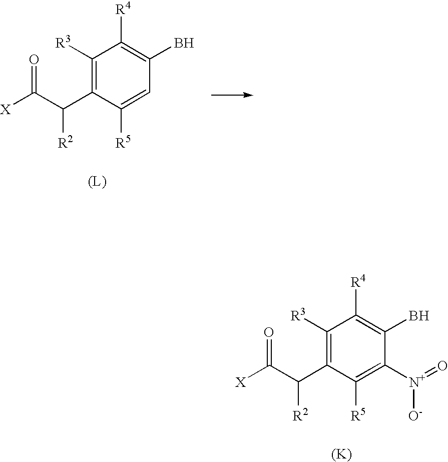

Compounds of formula (L) are known compounds or may be made from known compounds by known methods.

The compounds of formula (I) can be used to combat and control infestations of insect pests such as Lepidoptera, Diptera, Hemiptera, Thysanoptera, Orthoptera, Dictyoptera, Coleoptera, Siphonaptera, Hymenoptera and Isoptera and also other invertebrate pests, for example, acarine, nematode and mollusc pests. Insects, acarines, nematodes and molluscs are hereinafter collectively referred to as pests. The pests which may be combated and controlled by the use of the invention compounds include those pests associated with agriculture (which term includes the growing of crops for food and fibre products), horticulture and animal husbandry, companion animals, forestry and the storage of products of vegetable origin (such as fruit, grain and timber); those pests associated with the damage of man-made structures and the transmission of diseases of man and animals; and also nuisance pests (such as flies).

Examples of pest species which may be controlled by the compounds of formula (I) include: Myzus persicae (aphid), Aphis gossypii (aphid), Aphis fabae (aphid), Lygus spp. (capsids), Dysdercus spp. (capsids), Nilaparvata lugens (planthopper), Nephotettixc incticeps (leafhopper), Nezara spp. (stinkbugs), Euschistus spp. (stinkbugs), Leptocorisa spp. (stinkbugs), Frankliniella occidentalis (thrip), Thrips spp. (thrips), Leptinotarsa decemlineata (Colorado potato beetle), Anthonomus grandis (boll weevil), Aonidiella spp. (scale insects), Trialeurodes spp. (white flies), Bemisia tabaci (white fly), Ostrinia nubilalis (European corn borer), Spodoptera liaoralis (cotton leafworm), Heliothis virescens (tobacco budworm), Helicoverpa armigera (cotton bollworm), Helicoverpa zea (cotton bollworm), Sylepta derogata (cotton leaf roller), Pieris brassicae (white butterfly), Plutella xylostella (diamond back moth), Agrotis spp. (cutworms), Chilo suppressalis (rice stem borer), Locustamigratoria (locust), Chortiocetes tenninifera (ocust), Diabrotica spp. (rootworms), Panonychus ulmi (European red mite), Panonychus citri (citrus red mite), Tetranychus urticae (two-spotted spider mite), Tetranychus cinnabarinus (carmine spider mite), Phyllocoptruta oleivora (citrus rust mite), Polyphagotarsonemus latus (broad mite), Brevipalpus spp. (flat mites), Boophilus microplus (cattle tick), Dermacentor variabilis (American dog tick), Ctenocephalides felis (cat flea), Liriomyza spp. (leafminer), Musca domestica (housefly), Aedes aegypti (mosquito), Anopheles spp. (mosquitoes), Culex spp. (mosquitoes), Lucillia spp. (blowflies), Blaaella germanica (cockroach), Periplaneta americana (cockroach), Blatta orientalis (cockroach), tennites of the Mastotermitidae (for example Mastotermes spp.), the Kalotermitidae (for example Neoternes spp.), the Rhinotermitidae (for example Coptotermes formosanus, Reticuliternes flavipes, R. speratu, k virginicus, R. hesperus, and R. santonensis) and the Termitidae (for example Globitermes sulphureus), Solenopsis geminata (fire ant), Monomorium pharaonis (pharaoh's ant), Damalinia spp. and Linognathus spp. (biting and sucking lice), Meloidogyne spp. (root knot nematodes), Globodera spp. and Heterodera spp. (cyst nematodes), Pratylenchus spp. (lesion nematodes), Rhodopholus spp. (banana burrowing nematodes), Tylenchulus spp.(citrus nematodes), Haemonchus contortus (barber pole worm), Caenorhabditis elegans (vinegar eelworm), Trichostrongylus spp. (gastro intestinal nematodes) and Deroceras reticulatum (slug).

The compounds of formula (I) are also active fungicides and may be used to control one or more of the following pathogens: Pyricularia oryzae (Magnaporthe grisea) on rice and wheat and other Pyricularia spp. on other hosts; Puccinia recondita, Puccinia striiformis and other rusts on wheat, Puccinia hordei, Puccinia striiformis and other rusts on barley, and rusts on other hosts (for example turf, rye, coffee, pears, apples, peanuts, sugar beet, vegetables and ornamental plants); Erysiphe cichoracearum on cucurbits (for example melon); Erysiphe graminis (powdery mildew) on barley, wheat, rye and turf and other powdery mildews on various hosts, such as Sphaerotheca macularis on hops, Sphaerotheca fusca (Sphaerotheca fuliginea) on cucurbits (for example cucumber), Leveillula taurica on tomatoes, aubergine and green pepper, Podosphaera leucotricha on apples and Uncinula necator on vines; Cochliobolus spp., Helminthosporium spp., Drechslera spp. (Pyrenophora spp.), Rhynchosporium spp., Mycosphaerella graminicola (Septoria tritici) and Phaeosphaeria nodorum (Stagonospora nodorum or Septoria nodorum), Pseudocercosporella herpotrichoides and Gaeumannomyces graminis on cereals (for example wheat, barley, rye), turf and other hosts; Cercospora arachidicola and Cercosporidium personatum on peanuts and other Cercospora spp. on other hosts, for example sugar beet, bananas, soya beans and rice; Botrytis cinerea (grey mould) on tomatoes, strawberries, vegetables, vines and other hosts and other Botrytis spp. on other hosts; Alternaria spp. on vegetables (for example carrots), oil-seed rape, apples, tomatoes, potatoes, cereals (for example wheat) and other hosts; Venturia spp. (including Venturia inaequalis (scab)) on apples, pears, stone fruit, tree nuts and other hosts; Cladosporium spp. on a range of hosts including cereals (for example wheat) and tomatoes; Monilinia spp. on stone fruit, tree nuts and other hosts; Didymella spp. on tomatoes, turf, wheat, cucurbits and other hosts; Phoma spp. on oil-seed rape, turf, rice, potatoes, wheat and other hosts; Aspergillus spp. and Aureobasidium spp. on wheat, lumber and other hosts; Ascochyta spp. on peas, wheat, barley and other hosts; Stemphylium spp. (Pleospora spp.) on apples, pears, onions and other hosts; summer diseases (for example bitter rot (Glomerella cingulata), black rot or frogeye leaf spot (Botryosphaeria obtusa), Brooks fruit spot (Mycosphaerella pomi), Cedar apple rust (Gymnosporangium juniperi-virginianae), sooty blotch (Gloeodes pomigena), flyspeck (Schizothyrium pomi) and white rot (Botryosphaeria dothidea)) on apples and pears; Plasmopara viticola on vines; other downy mildews, such as Bremia lactucae on lettuce, Peronospora spp. on soybeans, tobacco, onions and other hosts, Pseudoperonospora humuli on hops and Pseudoperonospora cubensis on cucurbits; Pythium spp. (including Pythium ultimum) on turf and other hosts; Phytophthora infestans on potatoes and tomatoes and other Phytophthora spp. on vegetables, strawberries, avocado, pepper, ornamentals, tobacco, cocoa and other hosts; Thanatephorus cucumeris on rice and turf and other Rhizoctonia spp. on various hosts such as wheat and barley, peanuts, vegetables, cotton and turf; Scierotinia spp. on turf, peanuts, potatoes, oil-seed rape and other hosts; Sclerotium spp. on turf, peanuts and other hosts; Gibberella fujikuroi on rice; Colletotrichum spp. on a range of hosts including turf, coffee and vegetables; Laetisaria fuciformis on turf; Mycosphaerella spp. on bananas, peanuts, citrus, pecans, papaya and other hosts; Diaporthe spp. on citrus, soybean, melon, pears, lupin and other hosts; Elsinoe spp. on citrus, vines, olives, pecans, roses and other hosts; Verticillium spp. on a range of hosts including hops, potatoes and tomatoes; Pyrenopeziza spp. on oil-seed rape and other hosts; Oncobasidium theobromae on cocoa causing vascular streak dieback; Fusarium spp., Typhula spp., Microdochium nivale, Ustilago spp., Urocystis spp., Tilletia spp. and Claviceps purpurea on a variety of hosts but particularly wheat, barley, turf and maize; Ramularia spp. on sugar beet, barley and other hosts; post-harvest diseases particularly of fruit (for example Penicillium digitatum, Penicillium italicum and Trichoderna viride on oranges, Colletotrichum musae and Gloeosporium musarum on bananas and Botrytis cinerea on grapes); other pathogens on vines, notably Eutypa lata, Guignardia bidwellii, Phellinus igniarus, Phomopsis viticola, Pseudopeziza tracheiphila and Stereum hirsutum; other pathogens on trees (for example Lophodennium seditiosum) or lumber, notably Cephaloascus fragrans, Ceratocystis spp., Ophiostoma piceae, Penicillium spp., Trichodenna pseudokoningii, Trichoderma viride, Trichoderma harzianum, Aspergillus niger, Leptographium lindbergi and Aureobasidium pullulans; and fungal vectors of viral diseases (for example Polymyxa graminis on cereals as the vector of barley yellow mosaic virus (BYMV) and Polymyxa betae on sugar beet as the vector of rhizomania).

A compound of formula (I) may move acropetally, basipetally or locally in plant tissue to be active against one or more fungi. Moreover, a compound of formula (I) may be volatile enough to be active in the vapour phase against one or more fungi on the plant.

The invention therefore provides a method of combating and controlling insects, acarines, nematodes or molluscs which comprises applying an insecticidally, acaricidally, nematicidally or molluscicidally effective amount of a compound of formula (I), or a composition containing a compound of formula (I), to a pest, a locus of pest, or to a plant susceptible to attack by a pest, and a method of combating and controlling fungi which comprises applying a fungicidally effective amount of a compound of formula (I), or a composition containing a compound of formula (I), to a plant, to a seed of a plant, to the locus of the plant or seed, to soil or to any other growth medium (for example a nutrient solution). The compounds of formula (I) are preferably used against insects, acarines, nematodes or fungi.

The term “plant” as used herein includes seedlings, bushes and trees. Furthermore, the fungicidal method of the invention includes protectant, curative, systemic, eradicant and antisporulant treatments.

As fungicides, the compounds of formula (I) are preferably used for agricultural, horticultural and turfgrass purposes in the form of a composition.

In order to apply a compound of formula (I) as an insecticide, acaricide, nematicide or molluscicide to a pest, a locus of pest, or to a plant susceptible to attack by a pest, or, as a fungicide to a plant, to a seed of a plant, to the locus of the plant or seed, to soil or to any other growth medium, a compound of formula (I) is usually formulated into a composition which includes, in addition to the compound of formula (I), a suitable inert diluent or carrier and, optionally, a surface active agent (SFA). SFAs are chemicals which are able to modify the properties of an interface (for example, liquid/solid, liquid/air or liquid/liquid interfaces) by lowering the interfacial tension and thereby leading to changes in other properties (for example dispersion, emulsification and wetting). It is preferred that all compositions (both solid and liquid formulations) comprise, by weight, 0.0001 to 95%, more preferably 1 to 85%, for example 5 to 60%, of a compound of formula (I). The composition is generally used for the control of pests or fungi such that a compound of formula (I) is applied at a rate of from 0.1 g to 10 kg per hectare, preferably from 1 g to 6 kg per hectare, more preferably from 1 g to 1 kg per hectare.

When used in a seed dressing, a compound of formula (I) is used at a rate of 0.0001 g to 10 g (for example 0.001 g or 0.05 g), preferably 0.005 g to 10 g, more preferably 0.005 g to 4 g, per kilogram of seed.

In another aspect the present invention provides an insecticidal, acaricidal, nematicidal, molluscicidal or fungicidal composition comprising an insecticidally, acaricidally, nematicidally, molluscicidally or fungicidally effective amount of a compound of formula (I) and a suitable carrier or diluent therefor. The composition is preferably an insecticidal, acaricidal, nematicidal or fungicidal composition.

In a still further aspect the invention provides a method of combating and controlling pests or fungi at a locus which comprises treating the pests or fungi or the locus of the pests or fungi with an insecticidally, acaricidally, nematicidally, molluscicidally or fungicidally effective amount of a composition comprising a compound of formula (I). The compounds of formula (I) are preferably used against insects, acarines, nematodes or fungi.

The compositions can be chosen from a number of formulation types, including dustable powders (DP), soluble powders (SP), water soluble granules (SG), water dispersible granules (WG), wettable powders (WP), granules (GR) (slow or fast release), soluble concentrates (SL), oil miscible liquids (OL), ultra low volume liquids (UL), emulsifiable concentrates (C), dispersible concentrates (DC), emulsions (both oil in water (EW) and water in oil (EO)), micro-emulsions (ME), suspension concentrates (SC), aerosols, fogging/smoke formulations, capsule suspensions (CS) and seed treatment formulations. The formulation type chosen in any instance will depend upon the particular purpose envisaged and the physical, chemical and biological properties of the compound of formula (I).

Dustable powders (DP) may be prepared by mixing a compound of formula (I) with one or more solid diluents (for example natural clays, kaolin, pyrophyllite, bentonite, alumina, montmorillonite, kieselguhr, chalk, diatomaceous earths, calcium phosphates, calcium and magnesium carbonates, sulphur, lime, flours, talc and other organic and inorganic solid carriers) and mechanically grinding the mixture to a fine powder.

Soluble powders (SP) may be prepared by mixing a compound of formula (I) with one or more water-soluble inorganic salts (such as sodium bicarbonate, sodium carbonate or magnesium sulphate) or one or more water-soluble organic solids (such as a polysaccharide) and, optionally, one or more wetting agents, one or more dispersing agents or a mixture of said agents to improve water dispersibility/solubility. The mixture is then ground to a fine powder. Similar compositions may also be granulated to form water soluble granules (SG).

Wettable powders (WP) may be prepared by mixing a compound of formula (I) with one or more solid diluents or carriers, one or more wetting agents and, preferably, one or more dispersing agents and, optionally, one or more suspending agents to facilitate the dispersion in liquids. The mixture is then ground to a fine powder. Similar compositions may also be granulated to form water dispersible granules (WG).

Granules (GR) may be formed either by granulating a mixture of a compound of formula (I) and one or more powdered solid diluents or carriers, or from pre-formed blank granules by absorbing a compound of formula (I) (or a solution thereof, in a suitable agent) in a porous granular material (such as pumice, attapulgite clays, fuller's earth, kieselguhr, diatomaceous earths or ground corn cobs) or by adsorbing a compound of formula (I) (or a solution thereof, in a suitable agent) on to a hard core material (such as sands, silicates, mineral carbonates, sulphates or phosphates) and drying if necessary. Agents which are commonly used to aid absorption or adsorption include solvents (such as aliphatic and aromatic petroleum solvents, alcohols, ethers, ketones and esters) and sticking agents (such as polyvinyl acetates, polyvinyl alcohols, dextrins, sugars and vegetable oils). One or more other additives may also be included in granules (for example an emulsifying agent, wetting agent or dispersing agent).

Dispersible Concentrates (DC) may be prepared by dissolving a compound of formula (I) in water or an organic solvent, such as a ketone, alcohol or glycol ether. These solutions may contain a surface active agent (for example to improve water dilution or prevent crystallisation in a spray tank).

Emulsifiable concentrates (EC) or oil-in-water emulsions (EW) may be prepared by dissolving a compound of formula (I) in an organic solvent (optionally containing one or more wetting agents, one or more emulsifying agents or a mixture of said agents). Suitable organic solvents for use in ECs include aromatic hydrocarbons (such as alkylbenzenes or alkylnaphthalenes, exemplified by SOLVESSO 100, SOLVESSO 150 and SOLVESSO 200; SOLVESSO is a Registered Trade Mark), ketones (such as cyclohexanone or methylcyclohexanone) and alcohols (such as benzyl alcohol, furfuryl alcohol or butanol), N-alkylpyrrolidones (such as N-methylpyrrolidone or N-octylpyrrolidone), dimethyl amides of fatty acids (such as C₈-C₁₀ fatty acid dimethylamide) and chlorinated hydrocarbons. An EC product may spontaneously emulsify on addition to water, to produce an emulsion with sufficient stability to allow spray application through appropriate equipment. Preparation of an EW involves obtaining a compound of formula (I) either as a liquid (if it is not a liquid at room temperature, it may be melted at a reasonable temperature, typically below 70° C.) or in solution (by dissolving it in an appropriate solvent) and then emulsifiying the resultant liquid or solution into water containing one or more SFAs, under high shear, to produce an emulsion. Suitable solvents for use in EWs include vegetable oils, chlorinated hydrocarbons (such as chlorobenzenes), aromatic solvents (such as alkylbenzenes or alkylnaphthalenes) and other appropriate organic solvents which have a low solubility in water.

Microemulsions (ME) may be prepared by mixing water with a blend of one or more solvents with one or more SFAs, to produce spontaneously a thermodynamically stable isotropic liquid formulation. A compound of formula (I) is present initially in either the water or the solvent/SFA blend. Suitable solvents for use in MEs include those hereinbefore described for use in in ECs or in EWs. An ME may be either an oil-in-water or a water-in-oil system (which system is present may be determined by conductivity measurements) and may be suitable for mixing water-soluble and oil-soluble pesticides in the same formulation. An ME is suitable for dilution into water, either remaining as a microemulsion or forming a conventional oil-in-water emulsion.

Suspension concentrates (SC) may comprise aqueous or non-aqueous suspensions of finely divided insoluble solid particles of a compound of formula (I). SCs may be prepared by ball or bead milling the solid compound of formula (I) in a suitable medium, optionally with one or more dispersing agents, to produce a fine particle suspension of the compound. One or more wetting agents may be included in the composition and a suspending agent may be included to reduce the rate at which the particles settle. Alternatively, a compound of formula (I) may be dry milled and added to water, containing agents hereinbefore described, to produce the desired end product.

Aerosol formulations comprise a compound of formula (I) and a suitable propellant (for example n-butane). A compound of formula (I) may also be dissolved or dispersed in a suitable medium (for example water or a water miscible liquid, such as n-propanol) to provide compositions for use in non-pressurised, hand-actuated spray pumps.

A compound of formula (I) may be mixed in the dry state with a pyrotechnic mixture to form a composition suitable for generating, in an enclosed space, a smoke containing the compound.

Capsule suspensions (CS) may be prepared in a manner similar to the preparation of EW formulations but with an additional polymerisation stage such that an aqueous dispersion of oil droplets is obtained, in which each oil droplet is encapsulated by a polymeric shell and contains a compound of formula (I) and, optionally, a carrier or diluent therefor. The polymeric shell may be produced by either an interfacial polycondensation reaction or by a coacervation procedure. The compositions may provide for controlled release of the compound of formula (I) and they may be used for seed treatment. A compound of formula (I) may also be formulated in a biodegradable polymeric matrix to provide a slow, controlled release of the compound.

A composition may include one or more additives to improve the biological performance of the composition (for example by improving wetting, retention or distribution on surfaces; resistance to rain on treated surfaces; or uptake or mobility of a compound of formula (I)). Such additives include surface active agents, spray additives based on oils, for example certain mineral oils or natural plant oils (such as soy bean and rape seed oil), and blends of these with other bio-enhancing adjuvants (ingredients which may aid or modify the action of a compound of formula (I)).

A compound of formula (I) may also be formulated for use as a seed treatment, for example as a powder composition, including a powder for dry seed treatment (DS), a water soluble powder (SS) or a water dispersible powder for slurry treatment (WS), or as a liquid composition, including a flowable concentrate (FS), a solution (LS) or a capsule suspension (CS). The preparations of DS, SS, WS, FS and LS compositions are very similar to those of, respectively, DP, SP, WP, SC and DC compositions described above. Compositions for treating seed may include an agent for assisting the adhesion of the composition to the seed (for example a mineral oil or a film-forming barrier).

Wetting agents, dispersing agents and emulsifying agents may be surface SFAs of the cationic, anionic, amphoteric or non-ionic type.

Suitable SFAs of the cationic type include quaternary ammonium compounds (for example cetyltrimethyl ammonium bromide), imidazolines and amine salts.

Suitable anionic SFAs include alkali metals salts of fatty acids, salts of aliphatic monoesters of sulphuric acid (for example sodium lauryl sulphate), salts of sulphonated aromatic compounds (for example sodium dodecylbenzenesulphonate, calcium dodecylbenzenesulphonate, butylnaphthalene sulphonate and mixtures of sodium di-isopropyl- and tri-isopropyl-naphthalene sulphonates), ether sulphates, alcohol ether sulphates (for example sodium laureth-3-sulphate), ether carboxylates (for example sodium laureth-3-carboxylate), phosphate esters (products from the reaction between one or more fatty alcohols and phosphoric acid (predominately mono-esters) or phosphorus pentoxide (predominately di-esters), for example the reaction between lauryl alcohol and tetraphosphoric acid; additionally these products may be ethoxylated), sulphosuccinamates, paraffin or olefine sulphonates, taurates and lignosulphonates.

Suitable SFAs of the amphoteric type include betaines, propionates and glycinates.

Suitable SFAs of the non-ionic type include condensation products of alkylene oxides, such as ethylene oxide, propylene oxide, butylene oxide or mixtures thereof, with fatty alcohols (such as oleyl alcohol or cetyl alcohol) or with alkylphenols (such as octylphenol, nonylphenol or octylcresol); partial esters derived from long chain fatty acids or hexitol anhydrides; condensation products of said partial esters with ethylene oxide; block polymers (comprising ethylene oxide and propylene oxide); alkanolamides; simple esters (for example fatty acid polyethylene glycol esters); amine oxides (for example lauryl dimethyl amine oxide); and lecithins.

Suitable suspending agents include hydrophilic colloids (such as polysaccharides, polyvinylpyrrolidone or sodium carboxymethylcellulose) and swelling clays (such as bentonite or attapulgite).

A compound of formula (I) may be applied by any of the known means of applying pesticidal or fungicidal compounds. For example, it may be applied, formulated or unformulated, to the pests or to a locus of the pests (such as a habitat of the pests, or a growing plant liable to infestation by the pests) or to any part of the plant, including the foliage, stems, branches or roots, to the seed before it is planted or to other media in which plants are growing or are to be planted (such as soil surrounding the roots, the soil generally, paddy water or hydroponic culture systems), directly or it may be sprayed on, dusted on, applied by dipping, applied as a cream or paste formulation, applied as a vapour or applied through distribution or incorporation of a composition (such as a granular composition or a composition packed in a water-soluble bag) in soil or an aqueous environment.

A compound of formula (I) may also be injected into plants or sprayed onto vegetation using electrodynamic spraying techniques or other low volume methods, or applied by land or aerial irrigation systems.

Compositions for use as aqueous preparations (aqueous solutions or dispersions) are generally supplied in the form of a concentrate containing a high proportion of the active ingredient, the concentrate being added to water before use. These concentrates, which may include DCs, SCs, ECs, EWs, MEs SGs, SPs, WPs, WGs and CSs, are often required to withstand storage for prolonged periods and, after such storage, to be capable of addition to water to form aqueous preparations which remain homogeneous for a sufficient time to enable them to be applied by conventional spray equipment. Such aqueous preparations may contain varying amounts of a compound of formula (I) (for example 0.0001 to 10%, by weight) depending upon the purpose for which they are to be used.

A compound of formula (I) may be used in mixtures with fertilisers (for example nitrogen-, potassium- or phosphorus-containing fertilisers). Suitable formulation types include granules of fertiliser. The mixtures suitably contain up to 25% by weight of the compound of formula (I).

The invention therefore also provides a fertiliser composition comprising a fertiliser and a compound of formula (I).

The compositions of this invention may contain other compounds having biological activity, for example micronutrients or compounds having similar or complementary fungicidal activity or which possess plant growth regulating, herbicidal, insecticidal, nematicidal or acaricidal activity.

By including another fungicide, the resulting composition may have a broader spectrum of activity or a greater level of intrinsic activity than the compound of formula (I) alone. Further the other fungicide may have a synergistic effect on the fungicidal activity of the compound of formula (I).

The compound of formula (I) may be the sole active ingredient of the composition or it may be admixed with one or more additional active ingredients such as a pesticide, fungicide, synergist, herbicide or plant growth regulator where appropriate. An additional active ingredient may: provide a composition having a broader spectrum of activity or increased persistence at a locus; synergise the activity or complement the activity (for example by increasing the speed of effect or overcoming repellency) of the compound of formula (I); or help to overcome or prevent the development of resistance to individual components. The particular additional active ingredient will depend upon the intended utility of the composition. Examples of suitable pesticides include the following:

a) Pyrethroids, such as permethrin, cypermethrin, fenvalerate, esfenvalerate, deltamethrin, cyhalothrin (in particular lambda-cyhalothrin), bifenthrin, fenpropathrin, cyfluthrin, tefluthrin, fish safe pyrethroids (for example ethofenprox), natural pyrethrin, tetramethrin, s-bioallethrin, fenfluthrin, prallethrin or 5-benzyl-3-furylmethyl-(E)(1R,3S)-2,2-dimethyl-3-(2-oxothiolan-3-ylidenemethyl)cyclopropane carboxylate;

b) Organophosphates, such as, profenofos, sulprofos, acephate, methyl parathion, azinphos-methyl, demeton-s-methyl, heptenophos, thiometon, fenamiphos, monocrotophos, profenofos, triazophos, methamidophos, dimethoate, phosphamidon, malathion, chlorpyrifos, phosalone, terbufos, fensulfothion, fonofos, phorate, phoxim, pirimiphos-methyl, pirimiphos-ethyl, fenitrothion, fosthiazate or diazinon;

c) Carbamates (including aryl carbamates), such as pirimicarb, triazamate, cloethocarb, carbofuran, furathiocarb, ethiofencarb, aldicarb, thiofurox, carbosulfan, bendiocarb, fenobucarb, propoxur, methomyl or oxamyl;

d) Benzoyl ureas, such as diflubenzuron, triflumuron, hexaflumuron, flufenoxuron or chlorfluazuron;

e) Organic tin compounds, such as cyhexatin, fenbutatin oxide or azocyclotin;

f) Pyrazoles, such as tebufenpyrad and fenpyroximate;

g) Macrolides, such as avermectins or milbemycins, for example abamectin, emamectin benzoate, ivermectin, milbemycin, spinosad or azadirachtin;

h) Hormones or pheromones;

i) Organochlorine compounds such as endosulfan, benzene hexachloride, DDT, chlordane or dieldrin;

j) Amidines, such as chlordimeform or amitraz;

k) Fumigant agents, such as chloropicrin, dichloropropane, methyl bromide or metam;

l) Chloronicotinyl compounds such as imidacloprid, thiacloprid, acetamiprid, nitenpyram or thiamethoxam;

m) Diacylhydrazines, such as tebufenozide, chromafenozide or methoxyfenozide;

n) Diphenyl ethers, such as diofenolan or pyriproxifen;

o) Indoxacarb;

p) Chlorfenapyr; or

q) Pymetrozine.

In addition to the major chemical classes of pesticide listed above, other pesticides having particular targets may be employed in the composition, if appropriate for the intended utility of the composition. For instance, selective insecticides for particular crops, for example stemborer specific insecticides (such as cartap) or hopper specific insecticides (such as buprofezin) for use in rice may be employed. Alternatively insecticides or acaricides specific for particular insect species/stages may also be included in the compositions (for example acaricidal ovo-larvicides, such as clofentezine, flubenzimine, hexythiazox or tetradifon; acaricidal motilicides, such as dicofol or propargite; acaricides, such as bromopropylate or chlorobenzilate; or growth regulators, such as hydramethylnon, cyromazine, methoprene, chlorfluazuron or diflubenzuron).

Examples of fungicidal compounds which may be included in the composition of the invention are (E)-N-methyl-2-[2-(2,5-dimethylphenoxymethyl)phenyl]-2-methoxy-iminoacetamide (SSF-129), 4-bromo-2-cyano-N,N-dimethyl-6-trifluoromethyl-benzimidazole-1-sulphonamide, α-[N-(3-chloro-2,6-xylyl)-2-methoxy-acetamido]-γ-butyrolactone, 4-chloro-2-cyano-N,N-dimethyl-5-p-tolylimidazole-1-sulfonamide (IKF-916, cyamidazosulfamid), 3-5-dichloro-N-(3-chloro-1-ethyl-1-methyl-2-oxopropyl)₄-methylbenzamide (RH-7281, zoxamide), N-allyl-4,5,-dimethyl-2-trimethylsilylthiophene-3-carboxamide (MON65500), N-(1-cyano-1,2-dimethylpropyl)-2-(2,4-dichlorophenoxy)propionamide (AC382042), N-(2-methoxy-5-pyridyl)-cyclopropane carboxamide, acibenzolar (CGA245704), alanycarb, aldimorph, anilazine, azaconazole, azoxystrobin, benalaxyl, benomyl, biloxazol, bitertanol, blasticidin S, bromuconazole, bupirimate, captafol, captan, carbendazim, carbendazim chlorhydrate, carboxin, carpropamid, carvone, CGA41396, CGA41397, chinomethionate, chlorothalonil, chlorozolinate, clozylacon, copper containing compounds such as copper oxychloride, copper oxyquinolate, copper sulphate, copper tallate and Bordeaux mixture, cymoxanil, cyproconazole, cyprodinil, debacarb, di-2-pyridyl disulphide 1,1′-dioxide, dichlofluanid, diclomezine, dicloran, diethofencarb, difenoconazole, difenzoquat, diflumetorim, O,O-di-iso-propyl-S-benzyl thiophosphate, dimefluazole, dimetconazole, dimethomorph, dimethirimol, diniconazole, dinocap, dithianon, dodecyl dimethyl ammonium chloride, dodemorph, dodine, doguadine, edifenphos, epoxiconazole, ethirimol, ethyl(Z)-N-benzyl-N-([methyl(methyl-thioethylhdeneaminooxycarbonyl)amino]thio)-β-alaninate, etridiazole, famoxadone, fenamidone (RPA407213), fenarimol, fenbuconazole, fenfuram, fenhexamid (KBR2738), fenpiclonil, fenpropidin, fenpropimorph, fentin acetate, fentin hydroxide, ferbam, ferinzone, fluazinam, fludioxonil, flumetover, fluoroimide, fluquinconazole, flusilazole, flutolanil, flutriafol, folpet, fuberidazole, furalaxyl, furametpyr, guazatine, hexaconazole, hydroxyisoxazole, hymexazole, imazalil, imibenconazole, iminoctadine, iminoctadine triacetate, ipconazole, iprobenfos, iprodione, iprovalicarb (SZX0722), isopropanyl butyl carbamate, isoprothiolane, kasugamycin, kresoxim-methyl, LY186054, LY211795, LY248908, mancozeb, maneb, mefenoxam, mepanipyrim, mepronil, metalaxyl, metconazole, metiram, metiram-zinc, metominostrobin, myclobutanil, neoasozin, nickel dimethyldithiocarbamate, nitrothal-isopropyl, nuarimol, ofurace, organomercury compounds, oxadixyl, oxasuilfron, oxolinic acid, oxpoconazole, oxycarboxin, pefurazoate, penconazole, pencycuron, phenazin oxide, phosetyl-A1, phosphorus acids, phthalide, picoxystrobin (ZA1963), polyoxin D, polyram, probenazole, prochloraz, procymidone, propamocarb, propiconazole, propineb, propionic acid, pyrazophos, pyrifenox, pyrimethanil, pyroquilon, pyroxyfur, pyrrolnitrin, quaternary ammonium compounds, quinomethionate, quinoxyfen, quintozene, sipconazole (F-155), sodium pentachlorophenate, spiroxamine, streptomycin, sulphur, tebuconazole, tecloftalam, tecnazene, tetraconazole, thiabendazole, thifluzamid, 2-(thiocyanomethylthio)benzothiazole, thiophanate-methyl, thiram, timibenconazole, tolclofos-methyl, tolylfluanid, triadimefon, triadimenol, triazbutil, triazoxide, tricyclazole, tridemorph, trifloxystrobin (CGA279202), triforine, triflumizole, triticonazole, validamycin A, vapam, vinclozolin, zineb and ziram.

The compounds of formula (I) may be mixed with soil, peat or other rooting media for the protection of plants against seed-borne, soil-borne or foliar fungal diseases.

Examples of suitable synergists for use in the compositions include piperonyl butoxide, sesamex, safroxan and dodecyl imidazole.

Suitable herbicides and plant-growth regulators for inclusion in the compositions will depend upon the intended target and the effect required.

An example of a rice selective herbicide which may be included is propanil. An example of a plant growth regulator for use in cotton is PIX™.

Some mixtures may comprise active ingredients which have significantly different physical, chemical or biological properties such that they do not easily lend themselves to the same conventional formulation type. In these circumstances other formulation types may be prepared. For example, where one active ingredient is a water insoluble solid and the other a water insoluble liquid, it may nevertheless be possible to disperse each active ingredient in the same continuous aqueous phase by dispersing the solid active ingredient as a suspension (using a preparation analogous to that of an SC) but dispersing the liquid active ingredient as an emulsion (using a preparation analogous to that of an EW). The resultant composition is a suspoemulsion (SE) formulation.

The invention is illustrated by the following Examples:

### EXAMPLE 1

This Example illustrates the preparation of Compound No. 24.

Step 1—Preparation of methyl (4-hydroxyphenyl)acetate.

Hydrogen chloride was bubbled through a solution of (4-hydroxyphenyl) acetic acid (25 g, 0.16 mole) in methanol (100 ml) at room temperature. An exotherm resulted in the solution refluxing for about 10 minutes. The mixture was allowed to cool to room temperature and the solvent was evaporated in vacuo to afford methyl (4-hydroxyphenyl) acetate as a yellow oil (27.5 g) which crystallised on seeding, m.p. 46-52° C.

¹H NMR (CDCl₃) δ: 3.57(2H,s); 3.71(3H,s); 6.0(1H,b); 6.76 (2H,m); 7.10(2H,m)ppm.

Step 2—Preparation of methyl (4-hydroxy-3-nitrophenyl)acetate.

Nitric acid (69% by weight, 16M, 20 ml) was added dropwise to a solution of methyl(4-hydroxyphenyl)acetate (50.0 g, 0.3 mole) in acetic acid (500 ml), maintaining the temperature of the reaction below 15° C. by external cooling. (An induction period was observed for this reaction.) Once gas chromatographic analysis had confirmed that the reaction was complete, the mixture was carefully quenched into water (21) with vigorous stirring. An emulsion formed which subsequently crystallised. After filtration, washing with water and drying, the desired product was obtained as a yellow powder.

¹H NMR (CDCl₃) δ: 3.63(2H,s); 3.72(3H,s); 7.14(1H,d); 7.52(1H,dd); 8.02(1H,d); 10.5(1H,s)ppm.

Step 3—Preparation of methyl(3-amino-4-hydroxyphenyl)acetate.

Methyl(4-hydroxy-3-nitrophenyl)acetate (48.9 g, 0.23 mole) and 5% palladium on carbon were suspended in methanol and the resulting mixture was hydrogenated until all the starting material had been consumed. The reaction mixture was filtered to remove the catalyst and the filter-cake was washed with methanol. The combined filtrate and washings were concentrated in vacuo, affording methyl(3-amino-4-hydroxyphenyl)acetate as a solid (41.0 g).

¹H NMR (d₆-DMSO) δ: 3.51(2H,s); 4.45(2H,b); 6.20(1H,dd); 6.40(1H,d); 6.49(1H,d); 8.87(1H,b)ppm.

Step 4—Preparation of methyl [2-(2-chloro-6-fluorophenyl)benzoxazol-5-yl]acetate.

2-Chloro-6-fluorobenzoyl chloride (55.1 g, 0.287 mol) was added dropwise to a stirred mixture of methyl(3-amino hydroxyphenyl)acetate (51.95 g, 0.287 mol) and sodium bicarbonate (24.1 g, 0.287 mol) in 1,2-dimethoxyethane (750 ml) at such a rate that the temperature of the reaction was maintained below 22° C. Once the addition was complete the mixture was stirred for 2 hours, then poured into water and extracted with ethyl acetate. The organic extract was washed with brine, dried over anhydrous magnesium sulfate, filtered and the filtrate evaporated in vacuo. The resulting solid (44 g) was suspended in xylene (500 ml), para-toluene sulfonic acid (4.98 g, 0.0261 mol) added and the mixture refluxed (Dean and Stark receiver used) for 31 hours. The mixture was cooled to room temperature, and partititoned between ethyl acetate and water. The organic phase was washed with brine, dried over anhydrous magnesium sulfate, filtered and the filtrate evaporated in vacuo. The residue was further purified by column chromatography on silica gel, eluting with ethyl acetate:hexane 1:4 to give methyl[2-(2-chloro-6-fluorophenyl)benzoxazol-5-yl]acetate (23.4 g) as a pale yellow oil.

¹H NMR (CDCl₃) δ: 3.72(s,3H); 3.81(s,2H); 7.18(m,1H); 7.38(m,2H); 7.46(m,1H); 7.60(d,1H); 7.8(d,1H)ppm.

Step 5—Preparation of methyl 2-[2(2-chloro-6-fluorophenyl)benzoxazol-5-yl]propionate.

Lithium diisopropylamide (2.0M solution in hexanes, 10.85 ml, 0.0217 mol) was added dropwise to a vigorously stirred solution of methyl [2-(2-chloro-6-fluorophenyl)benzoxazol-5-yl]acetate (6.3 g, 0.0197 mol) in tetrahydrofuran (70 ml) at −70° C. Once the addition was complete the mixture was stirred for 1 hour at −70° C. Methyl iodide (8.39 g, 0.0591 mol) was added dropwise, at such a rate that the temperature was maintained below −65° C., and once the addition was complete the mixture was stirred at −65° C. for 15 minutes, and then allowed to warm slowly to room temperature and stirred for a further 2 hours. The mixture was poured into water, neutralised by addition of dilute aqueous hydrochloric acid and extracted with ethyl acetate. The organic extract was washed with brine, dried over anhydrous magnesium sulfate, filtered and the filtrate evaporated in vacuo. The residue was further purified by column chromatography on silica gel, eluting with ethyl acetate:hexane 1:9 to give methyl 2-[2-(2-chloro-6-fluorophenyl)benzoxazol-5-yl]propionate (5.6 g) as a pale yellow solid, m.p. 104-105° C.

¹H NMR (CDCl₃) δ: 1.6(d,3H); 3.70(s,3H); 3.90(m,1H); 7.18(m,1H); 7.39(m,2H); 7.45(m,1H); 7.6(dd,1H); 7.81(d,1H)ppm.

Step 6—Preparation of 2-[2-(2-chloro-6-fluorophenyl)benzoxazol-5-yl]propionic acid.

Hexamethyldisilane (0.852 g, 0.006 mol) and iodine (1.73 g, 0.007 mol) were added to a solution of methyl 2-[2-(2-chloro-6-fluorophenyl)benzoxazol-5-yl]propionate (2.16 g, 0.0065 mol) in toluene (20 ml) and the mixture heated to reflux for 6 hours. The mixture was cooled to room temperature, diluted with ethyl acetate and washed sequentially with water, saturated aqueous sodium thiosulfate solution and brine, dried over anhydrous magnesium sulfate, filtered and the filtrate evaporated in vacuo. The solid was triturated with hexane to give 2-[2-(2-chloro-6-fluorophenyl)-benzoxazol-5-yl]propionic acid (1.90 g).

¹H NMR (CDCl₃)δ: 1.62(d,3H); 3.92(q,1H); 7.2(m,1H); 7.45(m,3H); 7.62(d,1H); 7.88(br,1H)ppm.

Step 7—Preparation of N-(8-fluorocuinolin-4-yl)-2-[2-(2-chloro-6-fluorophenyl)benzoxazol-5-yl]propionamide.

1-(3-dimethylaminopropyl)-3-ethylcarbodiimide hydrochloride (0.334 g, 0.00174 mol) was added portionwise to a stirred mixture of 4-amino-8-fluoroquinoline (0.282 g, 0.00174 mol), 2-[2-(2-chloro-6-fluorophenyl)benzoxazol-5-yl]propionic acid (0.505 g, 0.00158 mol), and 1-hydroxy-7-azabenzotriazole (0.215 g, 0.00158 mol) in N,N-dimethylformamide (8 ml) and the mixture stirred at room temperature for 5 hours. The reaction mixture was poured into ice/water, and the precipitated solid collected by filtration, taken up in ethyl acetate and washed with brine. The organic phase was dried over anhydrous magnesium sulfate, filtered and the filtrate evaporated in vacuo. The residue was further purified by column chromatography on silica gel, eluting with ethyl acetate:hexane 1:1 to afford a pale yellow gum. Trituration with hexane/diethyl ether gave N-(8-fluoroquinolin-4-yl)-2-[2-(2-chloro-6-fluorophenyl)benzoxazol-5-yl]propionamide (0.326 g) as a colourless solid.

### EXAMPLE 2

This Example illustrates the preparation of Compound No. 27.

Lithium bis(trimethylsilyl)amide (1.0M solution in tetrahydrofuran, 1.25 ml) was added to a solution of N-(8-fluoroquinolin-4-yl)-2-[2-(2-chloro-6-fluorophenyl)benzoxazol-5-yl]propionamide (0.482 g, 0.001 mol) in tetrahydrofuran (5 ml) and the mixture stirred at room temperature for 30 minutes. Chloromethyl ethyl ether (0.298 g, 0.003 mol) was added and the mixture stirred at room temperature for 1 hour. The mixture was poured into saturated aqueous sodium bicarbonate solution and extracted with ethyl acetate. The organic extract was washed with brine, dried over anhydrous magnesium sulfate, filtered and the filtrate evaporated in vacuo. The residue was further purified by column chromatography on silica gel, eluting with ethyl acetate: dichloromethane 1:4 to afford N-ethoxymethyl-N-(8-fluoroquinolin-4-yl)-2-[2-(2-chloro-6-fluorophenyl)benzoxazol-5-yl]propionamide (0.21 g) as a pale orange gum.

### EXAMPLE 3

This Example illustrates the preparation of Compound No. 1.

Oxalyl chloride (0.907 g, 0.00715 mol) was added dropwise to a solution of 2-[2-(2-chloro-6-fluorophenyl)benzoxazol-5-yl]propionic acid (1.90 g, 0.00596 mol) and N,N-dimethylformamide (2 drops) in dichloromethane (20 ml) and the mixture stirred at room temperature for 90 minutes. The solvent was removed in vacuo, and the residue taken up in 1,2-dichloroethane (20 ml) and the mixture heated to reflux. A solution of 5-amino-4-chloro-3-ethylisothiazole (1.13 g, 0.00693 mol) in 1,2-dichloroethane (5 ml) was added dropwise, and once the addition was complete the mixture was refluxed for 2 hours. The mixture was cooled to room temperature and the solvent evaporated in vacuo. The residue was partitioned between ethyl acetate and water, and the organic phase washed with saturated aqueous sodium bicarbonate solution, dried over anhydrous magnesium sulfate, filtered and the filtrate evaporated in vacuo. The residue was further purified by column chromatography on silica gel, eluting with ethyl acetate:hexane 1:3 to afford N-(4-chloro-3-ethylisothiazol-5-yl)-2-[2-(2-chloro-6-fluorophenyl)benzoxazol-5-yl]propionamide (1.92 g) as a cream solid, m.p. 178-179° C.

### EXAMPLE 4

This Example illustrates the preparation of Compound No. 21.

N,O-Bis(trimethylsilyl)acetamide (0.244 g, 0.0012 mol) was added to a solution of N-(4-chloro-3-ethylisothiazol-5-yl)-2-[2-(2-chloro-6-fluorophenyl)benzoxazol-5-yl]propionamide (0.463 g, 0.001 mol) in dichloromethane and the mixture stirred at room temperature for 30 minutes. Chloromethyl ethyl ether (0.189 g, 0.002 mol) was added and the mixture stirred at room temperature for 42 hours. The mixture was poured into saturated aqueous sodium bicarbonate solution and extracted with dichloromethane. The organic extract was washed with brine, dried over anhydrous magnesium sulfate, filtered and the filtrate evaporated in vacuo. The residue was further purified by column chromatography on silica gel, eluting with ethyl acetate:hexane 1:3 to afford N-(4-chloro-3-ethyhisothiazol-5-yl)-N-ethoxymethyl-2-[2-(2-chloro-6-fluorophenyl)benzoxazol-5-yl]propionamide (0.294 g).

### EXAMPLE 5

This Example Illustrates the Preparation of Compound No. 11.

Step 1—Preparation of 2-(4-hydroxy-3-nitrophenyl)propionic acid.

Ferric nitrate nonahydrate (48.67 g, 0.12 mol) was added to a solution of 2-(4-hydroxyphenyl)propionic acid (20.0 g, 0.12 mol) in ethanol (200 ml) and the mixture stirred at room temperature for 24 h. The mixture was poured into dilute aqueous hydrochloric acid (400 ml) and extracted with ethyl acetate. The organic extract was washed with dilute aqueous hydrochloric acid and brine, dried over anhydrous magnesium sulfate, filtered and the filtrate evaporated in vacuo. The residue was triturated with hexane to give (23.84 g) as a yellow solid, m.p. 136-138° C.

¹H NMR (d₆-DMSO) δ: 1.3(d,3H); 3.65(m,1H); 7.03(d,1H); 7.41(dd,1H); 7.71(d,1H); 10.85s,1H)ppm.

Step 2—Preparation of N-(4-chloro-3-ethylisothiazol-5-yl)-2-(4-hydroxy-3-nitrophenyl)propionamide.

Oxalyl chloride (25.9 g, 0.204 mol) was added dropwise to a solution of 2-(4 hydroxy-3-nitrophenyl)propionic acid (39.0 g, 0.186 mol) and N,N-dimethylformamide (0.4 ml) in dichloromethane (250 ml) and the mixture stirred at room temperature for 1 hour and then at 35° C. for 30 minutes. The volatiles were removed in vacuo, and the residue taken up in 1,2-dichloroethane (450 ml) and heated to 84° C. A solution of 5-amino-4-chloro-3-ethylisothiazole (31.9 g, 0.050 mol) in 1,2-dichloroethane (150 ml) was added dropwise, and once the addition was complete the mixture was refluxed for 2 hours. The mixture was cooled to room temperature and the solvent evaporated in vacuo. The residue was taken up in ethyl acetate, washed with brine, dried over anhydrous magnesium sulfate, filtered and the filtrate was evaporated in vacuo. The residue was triturated with hexane to give N-(4-chloro-3-ethylisothiazol-5-yl)-2-(4-hydroxy-3-nitrophenyl)propionamide (48.0 g).

¹H NMR (CDCl₃) δ: 1.27(t,3H); 1.67(d,3M); 2.73(q,21); 3.89(m,1H); 7.2(d,1H); 7.63(dd,1H); 8.1(d,1H); 8.2(br,1H); 10.6(s,1H)ppm.

Step 3—Preparation of N-(4-chloro-3-ethylisothiazol-5-yl).2-(3-amino-4-hydroxyphenyl)propionamide.

N-(4-chloro-3-ethylisothiazol-5-yl)-2-(4-hydroxy-3-nitrophenyl)propionamide (30.0 g, 0.084 mole) and 1% platinum on carbon (15 g) were suspended in N,N-dimethylformamide and the resulting mixture was hydrogenated at 15 bar and stirred at 30° C. for 5 hours. The reaction mixture was filtered to remove the catalyst and the filtrate concentrated in vacuo. The residue was triturated with a mixture of dichloromethane and hexane to give N-(4-chloro-3-ethylisothiazol-5-yl)-2-(3-amino-4-hydroxyphenyl)propionamide (17.6 g).

¹H NMR (d₆-DMSO) δ: 1.25(t,3H); 1.52(d,3H); 2.7(q,2H); 3.9(m,1H); 6.57(dd,1H); 6.69(d,1H); 6.76(d,1H); 9.52(br,1H)ppm.

Step 4—Preparation of N-(4-chloro-3-methylisothiazol-5-yl)-2-[2-(2,3-dichlorophenyl)benzoxazol-5-yl]propionamide.

2,3-Dichlorobenzoyl chloride (0.32 g, 0.0015 mol) was added to a chilled (ice-bath) solution of N-(4-chloro-3-ethylisothiazol-5-yl)-2-(3-amino-4-hydroxyphenyl)propionamide (0.50 g, 0.0015 mol) and pyridine (0.12 ml) in N,N-dimethylacetamide (5 ml) and the mixture stirred for 3 hours. The cooling bath was removed and the mixture was allowed to warm to room temperature overnight. The mixture was poured into icetwater and the precipitate collected by filtration and dried in vacuo. The product obtained was suspended in 1,1,2,2-tetrachloroethane (7 ml), para-toluenesulfonic acid (0.073 g, 0.0004 mol) added and the mixture heated at reflux overnight. The mixture was diluted with chloroform and washed with brine. The organic extract was evaporated in vacuo and the residue further purified by column chromatography on silica gel, eluting initially with hexane and then with ethyl acetate:hexane 3:7 to give N-(4-chloro-3-methylisothiazol-5-yl)-2-[2-(2,3-dichlorophenyl)benzoxazol-5-yl]propionamide as a cream solid, m.p. 158-161° C.

### EXAMPLE 6

This Example illustrates the pesticidal/insecticidal properties of compounds of formula (I). The activities of individual compounds of formula (I) were determined using a variety of pests. The pests were treated with a liquid composition containing 500 parts per million (ppm) by weight of a compound of formula (I). Each composition was made by dissolving the compound in an acetone and ethanol (50:50 by volume) mixture and diluting the solution with water containing 0.05% by volume of a wetting agent, SYNPERONIC NP8, until the liquid composition contained the required concentration of the compound. SYNPERONIC is a registered trade mark.

The test procedure adopted with regard to each pest was essentially the same and comprised supporting a number of the pests on a medium, which was usually a substrate, a host plant or a foodstuff on which the pests feed, and treating either or both the medium and the pests with a composition. Pest mortality was assessed usually between two and five days after treatment.

In each test against peach potato aphids (Myzus persicae), Chinese cabbage leaves were infested with aphids, the infested leaves were sprayed with a test composition and pest mortality was assessed after three days.

Similar tests were conducted against, independently, two-spotted spider mites (Tetranychus urticae), fruit flies (Drosophila melanogaster), tobacco budworms (Heliothis virescens), diamond back moth (Plutella xylostella) and corn root worm (Diabrotica balteata).

Tests were also conducted against root knot nematodes (Meloidogyne incognita) using an in vitro test in which nematodes were suspended in a liquid composition which had been prepared as described above except that it contained a concentration of 12.5 ppm by weight of a compound of formula (I) and it contained no SYNPERONIC NP8.

Results from these tests are displayed in Table 3, in which each mortality (score) is designated as 9, 5 or 0 wherein 9 indicates 80-100% mortality, 5 indicates 40-79% mortality and 0 indicates less than 40% mortality; and Mp represents Myzus persicae; Db represents Diabrotica balteata; Hv represents Heliothis virescens; Px represents Plutella xylostella; Mi represents Meloidogyne incognita; and Dm represents Drosophila melanogaster.

TABLE 3

| Compound | | | | | | |
|---|---|---|---|---|---|---|
| Number | Mp | Db | Hv | Px | Mi | Dm |
| 1 | 9 | 9 | 9 | 9 | 0 | 9 |
| 2 | 0 | 9 | 9 | 9 | 5 | 0 |
| 3 | 0 | 0 | 9 | 5 | 0 | 0 |
| 4 | 9 | 5 | 9 | 9 | 5 | 0 |
| 5 | 9 | 9 | 9 | 9 | 0 | 9 |
| 6 | 9 | 9 | 5 | 9 | 0 | 9 |
| 7 | 9 | 9 | 9 | 9 | 0 | 9 |

1. A compound of formula (I): 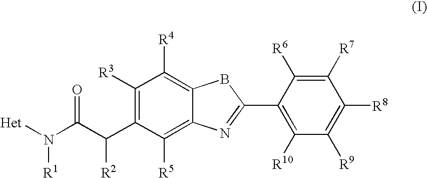
where B is O or S; Het is a heterocycle selected from heterocycles (a), (b), (c), (d), (e), (f), (g) and (h) in each of which the arrow shows the point of attachment to N of formula (I); 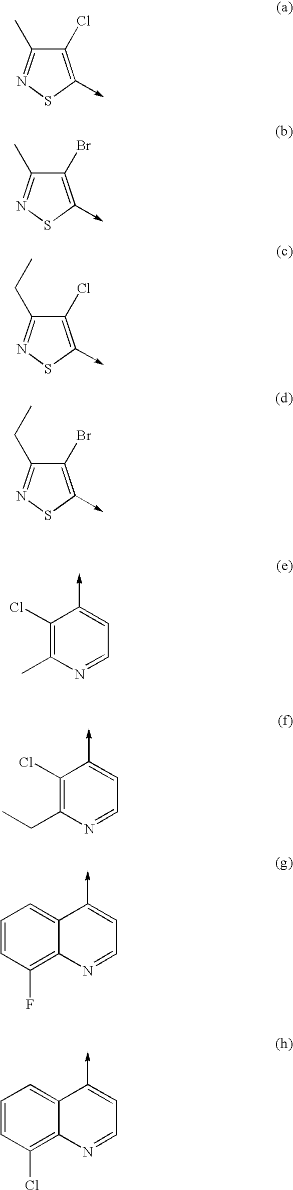
R¹ is hydrogen, C₁₋₂ alkyl, (C₁₋₆)alkoxymethyl or propargyl; R² is hydrogen, methyl or fluoro; R³, R⁴ and R⁵ are, independently, hydrogen, halogen, C₁₋₂ alkyl, C₁₋₂ alkoxy or C₁₋₂ haloalkyl; R⁶ and R¹⁰ are, independently, hydrogen, halogen, C₁₋₃ alkyl, C₁₋₂ haloalkyl, C₁₋₂ alkoxy, nitro, cyano, C₁₋₂ haloalkoxy, C₁₋₈ alkylthio, C₁₋₆ alkylsulfinyl, C₁₋₆ alkylsulfonyl, amino, C₁₋₃ alkylamino or di(C₁₋₃)alkylamino, provided that at least one of R⁶ and R¹⁰ is not hydrogen; R⁷, R⁸ and R⁹ are, independently, hydrogen, halogen, C₁₋₆ alkyl, C₂₋₆ alkenyl, C₂₋₆ alkynyl, C₁₋₆ haloalkyl, C₁₋₆ alkoxy(C₁₋₆)alkyl, C₁₋₆ alkoxy, C₁₋₆ alkoxy(C₁₋₆)alkoxy, C₂₋₆ alkynyloxy, C₃₋₆ cycloalkyl, nitro, cyano, C₁₋₆ haloalkoxy, C₂₋₆ haloalkenyloxy, S(O)ₚR¹¹, OSO₂R¹², NR¹³SO₂R¹⁴, NR¹⁵R¹⁶, NR¹⁷COR¹⁸, COR¹⁹, SiR²⁰R²¹R²², SCN, optionally substituted aryl or optionally substituted heteroaryl; R¹¹, R¹² and R¹⁴ are, independently, C₁₋₆ alkyl, C₁₋₆ haloalkyl or optionally substitituted aryl; R¹³ and R¹⁷ are, independently, hydrogen or C₁₋₂ alkyl; R¹⁵ and R¹⁶ are, independently, hydrogen or C₁₋₃ alkyl; or R¹⁵ and R¹⁶ together with the N atom to which they are attached form a five or six-membered optionally substituted heterocyclic ring which may contain a further heteroatom selected from O and S; R¹⁸ and R¹⁹ are, independently, hydrogen, C₁₋₆ alkyl, C₁₋₆ alkoxy, optionally substituted aryl, optionally substituted heteroaryl or NR²³R²⁴; R²⁰, R²¹ and R²² are, independently, C₁₋₄ alkyl or aryl; R²³ and R²⁴ are, independently, hydrogen or C₁₋₃ alkyl; or R²³ and R²⁴ together with the N atom to which they are attached form a five or six-membered optionally substituted heterocyclic ring which may contain a further heteroatom selected from O and S; and p is 0, 1 or 2; provided that when Het is a heterocycle selected from heterocycles (a), (b), (c) and (d); and R¹ is hydrogen, C₁₋₂ alkyl, (C₁₋₂)alkoxymethyl or propargyl; and R² is hydrogen; and R³, R⁴ and R⁵ are each hydrogen; then the moiety (M) where the arrow shows the point of attachment to the benzo-fused ring system of formula (I) 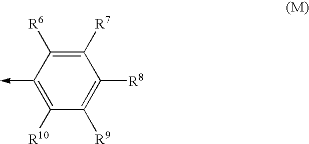
is not 2-Br—C₆H₄, 2-Cl—C₆H₄, 2,3-diCl—C₆H₃, 2,4-diCl—C₆H₃, 2,5-diCl—C₆H₃, 2,6-diCl—C₆H₃, 2,4,6-triCl—C₆H₂, C₆Cl₅, 2-Cl-4-F—C₆H₃, 2-Cl-6-F—C₆H₃, 4-Cl-2,5-diF—C₆H₂, 2-Cl-4-NO₂—C₆H₃, 2-Cl-4-CF₃—C₆H₃, 2-Cl-6—CF₃—C₆H₃, 2-Cl-4-methanesulfonyl-C₆H₃, 2,4-diCl-5-F—C₆H₂, 2-F—C₆H₄, 2,3-diF—C₆H₃, 2,4-diF—C₆H₃, 2,5-diF—C₆H₃, 2,6-diF—C₆H₃, 2,3,4-triF—C₆H₂, 2,3,5-triF—C₆H₂, 2,3,6-triF—C₆H₂, 2,4,6-triF—C₆H₂, 2,3,4,5-tetraF—C₆H, C₆F₅, 2-F-3-CF₃—C₆H₃, 2-F-4-CF₃—C₆H₃, 2-F5-CF₃—C₆H₃, 2-F-6-CF₃—C₆H₃, 4-F-2-CF₃—C₆H₃, 5-F-2-CF₃—C₆H₃, 2-CN—C₆H₄, 2-C₂H₅O—C₆H₄, 2-C₂H₅—C₆H₄, 2-CH₃O—C₆H₄, 2,6-diCH₃O—C₆H₃, 2-CH₃—C₆H₄, 2,3-diCH₃—C₆H₃, 2,4-diCH₃—C₆H₃, 2,5-diCH₃—C₆H₃, 2,6-diCH₃—C₆H₃, 2,4,6-triCH₃—C₆H₂, 2-NO₂—C₆H₄, 4-methanesulfonyl-2-nitrophenyl or 2-trifluoromethylphenyl.

2. A compound of formula (I) as claimed in claim 1 where R⁶ and R¹⁰ are, independently, hydrogen, halogen, C₁₋₃ alkyl, C₁₋₂ haloalkyl, C₁₋₂ alkoxy, nitro, cyano, C₁₋₂ haloalkoxy, C₁₋₂alkylthio, amino, C₁₋₃ alkylamino or di(C₁₋₃)alkylamino, provided that at least one of R⁶ and R¹⁰ is not hydrogen; and R⁷, R⁸ and R⁹ are, independently, hydrogen, halogen, C₁₋₆ alkyl, C₂₋₆ alkenyl, C₂₋₆ alkynyl, C₁₋₆ haloalkyl, C₁₋₆ alkoxy(C₁₋₆)alkyl, C₁₋₆ alkoxy, C₃₋₆ cycloalkyl, nitro, cyano, C₁₋₆ haloalkoxy, S(O)ₚR¹¹, OSO₂R¹², NR¹³SO₂R¹⁴, NR¹⁵R¹⁶, NR¹⁷COR¹⁸, COR¹⁹, SiR²⁰R²¹R²², SCN, optionally substituted aryl or optionally substituted heteroaryl.

3. A compound of formula (I) as claimed in claim 1 where B is O.

4. A compound of formula (I) as claimed in claim 1 where Het is a heterocycle selected from heterocycles (a), (c), (f) and (g).

5. A compound of formula (I) as claimed in claim 1 where R¹ is hydrogen, C₁₋₂ alkyl or (C₁₋₆) alkoxymethyl.

6. A compound of formula (I) as claimed in claim 1 where R² is hydrogen or methyl.

7. A compound of formula (I) as claimed in claim 1 where R³, R⁴ and R⁵ are each, independently, hydrogen or halogen.

8. An insecticidal, acaricidal, molluscicidal or nematicidal composition comprising an insecticidally, acaricidally, molluscicidally or nematicidally effective amount of a compound of formula (I) as claimed in claim 1 and a carrier or diluent therefor.

9. A method of combating and controlling insects, acarines, nematodes or molluscs which comprises applying to a pest, to a locus of a pest, or to a plant susceptible to attack by a pest an insecticidally, acaricidally, nematicidally or molluscicidally effective amount of a compound of formula (I) as claimed in claim 1.

10. A method of combating and controlling insects, acarines, nematodes or molluscs which comprises applying to a pest, to a locus of a pest, or to a plant susceptible to attack by a pest an insecticidally, acaricidally, nematicidally or molluscicidally effective amount of a composition as claimed in claim 8.
